(19) United States
(12) Patent Application Publication
(10) Pub. No.: US 2022/0383379 A1
(43) Pub. Date: Dec. 1, 2022

(54) APPLIANCE NETWORK WITH A SMART CONTROL, HOST MULTI-FUNCTION AND EXTERNAL APPLIANCE WITH FOOD CONTAINERS AND METHODS

(71) Applicant: Edward P. Espinosa, Madrid (ES)

(72) Inventor: Edward P. Espinosa, Madrid (ES)

(73) Assignee: Jeffrey S. Melcher, Boca Raton, FL (US)

(21) Appl. No.: 17/884,854

(22) Filed: Aug. 10, 2022

Related U.S. Application Data
(63) Continuation of application No. 15/911,682, filed on Mar. 5, 2018.
(60) Provisional application No. 62/484,502, filed on Apr. 12, 2017.
(60) Provisional application No. 62/467,315, filed on Mar. 6, 2017.

(51) Int. Cl. G06Q 30/06; G06Q 10/08; G06Q 50/12

(52) U.S. Cl. CPC G06Q 30/0621; G06Q 10/0875; G06Q 50/12; G06Q 10/083

(57) ABSTRACT

An appliance or compact multi-function appliance for use with a perishable food, product or container that are configured to track, monitor and report food freshness and cooking status and to order, purchase, process payments and provide home delivery for a product.

### FIELD OF THE INVENTION

The present invention discloses an appliance or compact multi-function appliance for use with a perishable food, product or container (“product”) that are configured to track, monitor and report food freshness and cooking status; preserve, disinfect, prepare and cook a product; and to order, purchase, process payments and provide home delivery for a product.

The present invention also relates to a method and system for manufacturing, packaging, displaying, selling, marketing, ordering, purchasing and processing payments for a product with container technologies for use with an appliance such as a smartphone, smart speaker, and smart refrigerator, as described herein.

Also disclosed is a method and system for the use of multiple brand logos or names and communicative indicia on a container or product packaging wherein at least one of the brand logos or names (such as a secondary or tertiary brand logo or name) can be used to represent unique, different or distinct products, services or benefits from the primary brand logo or name. Secondary or tertiary brand logos or names can communicate to consumers either an enhanced, unexpected or unseen use or benefit for a product, a container technology that can include a benefit for ordering, purchasing, processing a payment, providing a product delivery or for providing recipes and multiple cooking times for an appliance network or for a subscription service for said products and services.

The complete disclosure of U.S. Pat. No. 9,563,833 is incorporated herein by reference.

### BACKGROUND OF THE INVENTION

Current food, container and appliance food storage, cooking, marketing, ordering, purchasing, payment processing and delivery technologies and services are fragmented, inefficient and wasteful. Essentially, if a technology is difficult, time consuming or incompatible, every participant in the food, consumer good, appliance and product ordering chain (manufacturers, retailers, and consumers) will not benefit. There is a need for a more integrated and efficient food, container, appliance and product inventory management, ordering and payment processing ecosystem.

It is important that manufacturers and retailers display product packaging to provide the simplest and most effective communication indicia and text possible. Product containers are becoming increasingly more sophisticated incorporating container technologies to allow consumers to use said product containers as conventional storage containers or, if consumers are effectively informed, to use container technologies with appliances and services. Providing effective communicative indicia and text regarding benefits, uses and advantages regarding container technologies is challenging due to technological advances and to the limited space provided on a container or a readable technology.

### SUMMARY OF THE INVENTION

Objectives of the present invention include providing an appliance or multi-function appliance for use with products, containers and technologies to provide more efficient methods to track, monitor and report product freshness; facilitate cooking; and to order, purchase, process payments and provide delivery of products.

As used herein, a container can be any receptacle such as a sealable or resealable receptacle or closure for use with a container to hold a product which can be constructed to hold storage environments such as gas, vacuum, pressure or vented environments with said containers not being destroyed by said applications. Containers are designed for use with an appliance or multi-function appliance and include combined functions or modes as described herein; which can operate individually, in combination or in sequence with each or with other modes or appliances or in connection and communication with appliances and services (“container”).

A container with a container technology can utilize wireless technologies and combinations thereof. Radio frequency identification (“RFID”) is a wireless communication technology that can use a contactless integrated circuit card (“ic”) or tag. Near field communication (“NFC”) is a similar concept to RFID and can use a contactless is card or tag. However, NFC can differ from RFID because some RFID devices enable communication at a distance of several meters whereas NFC devices have antennas which can be brought within several centimeters from each other to communicate. For example, NFC can be used as a wireless technology to allow devices to wirelessly communicate with one another over close ranges. Typical distances can be several centimeters and communicating can also include waving or physical contact such as tapping. Current NFC communication can include contactless transactions and data exchange, among others. NFC is a short range wireless connectivity technology that can also allow the exchange of various types of information, such as text, numbers, pictures, cooking instructions, product and financial information or digital authorizations between two NFC enabled devices that can include interfaces such as smartphones, watches, wearables, appliances and containers and a compatible smart card or reader in close proximity. Li-Fi (Light Fidelity) is a bidirectional high speed and fully networked wireless communication technology similar to Wi-Fi which can also be incorporated into an appliance or and interface by incorporating Li-Fi enabling chips, software and hardware and with each appliance constructed with an indicator or Li-Fi light connected to or in communication with the interior of and exterior of an appliance to allow the sharing of data. Li-Fi is wireless and uses visible light communication or infra-red and near ultraviolet (instead of radio frequency waves) spectrum, part of optical wireless communications technology which can be used to complement other data communication networks.

A feature of NFC communication (as with related RFID) in one example includes passive communication capabilities. Passive communication can comprise of an active “interrogator” device (“reader”) that transmits a signal that is inductively harvested to power the passive interrogated device. The transmitted signal can also include a constant wave portion. The tag can respond to the interrogator by reflecting back a modulated version of the constant wave portion of the transmitted signal. Passive communication enables communication with otherwise unpowered tags for applications such as inventory management.

The technologies disclosed herein for use with a product, a container and technology and with interfaces or appliances, can include wireless or wired appliances for use with a wired or wireless appliance or device. In one embodiment, a wireless appliance can include: a power supply; an RFID/NFC or wireless interface configured to harvest energy radiated from an external interrogator appliance or device and to receive configuration data without receiving power from the power supply; a memory structure configured to store the received configuration data; a processor; and a non-transitory computer readable medium. In one application, the non-transitory computer readable medium includes one or more instructions configured to, when executed by the processor, cause the wireless device to retrieve the received configuration data from the memory structure, and configure the wireless appliance based on the received configuration data.

In another embodiment, a wireless appliance can include: a power supply; an RFID/NFC or wireless interface configured to utilize energy radiated from an external interrogator appliance, and to transmit one or more backup data without receiving power from the power supply; a memory structure configured to store the one or more backup data; a processor; and a non-transitory computer readable medium. In one application, the non-transitory computer readable medium can include one or more instructions configured to, when executed by the processor, cause the appliance to update the one or more backup data stored within the memory structure.

In another embodiment, backup data can comprise one or more of: user data;

marketing material; product information, product ordering, purchasing and payment processing data, technology and information; container and appliance applications; recipes and cooking instructions; chemical, gas or volatile organic compound signatures or profiles and threshold levels and any other information described herein including user contact data (user address, bank, credit/debit card or third party payment information), appliance specific data and can include network or appliance biometric access or authorizations using facial, eye/iris, fingerprint, palm or voice recognition connected to or in communication with an appliance and operating systems for product ordering, purchasing and payment processing or delivery and data and network configuration data, authorization and access. In certain situations, the one or more backup data can be encrypted. In another embodiment, the memory structure can comprise a removable or non-removable secure or non-secure element. Wireless and similar enabled appliances herein can function as either an initiator or target depending upon the user desired functionality and application.

Smartphones, smart speakers, beacons, appliances, containers, tablet terminals and other user interfaces can be equipped with RFID/NFC and wireless modules. RFID/NFC can operate using both passive and active communication. In passive communication, a reader/writer communicates with a contactless ic card or tag without a power source. In active communication, two devices both incorporating power sources communicate with each other by alternately acting as an initiator and a target.

An RFID/NFC enabled appliance can use a reader/writer function to read contactless ic cards. With NFC, an NFC antenna of one of the appliances is required to be brought within the communicable distance of an NFC antenna of the other appliance. However, with NFC it is possible to access a contactless ic card or tag having no power source to read or write data and it is only necessary to bring the appliance in close proximity to start or finish communication or exchange. This application allows for the use of containers, smartphones, smart speakers, beacons, products and appliances to perform transactions and exchanges with, for example, containers, smartphones, consumer and electronic devices, interfaces and appliances. RFID/NFC, as well as hybrid tags, and wireless technologies can allow a single appliance or container to operate in more than one mode. For example, a smartphone can be capable of functioning (a) in a card emulation mode to facilitate product ordering and payment in a credit or debit card transaction; and (b) in reader mode, to facilitate reading a container or other smart/sensor tags or other appliances. It is also possible for the technologies described herein to be capable of peer to peer information exchange.

As used herein, a container technology can include any technology that communicates, interacts, tracks, monitors, reports, benefits, orders, purchases, processes a payment or service or delivery or enhances the use, access, understanding, storage or appearance of a product such as a user interface, consumer or electronic device, good or appliance which can be placed, attached, affixed to or connect to or communicate with a product, a container or an appliance such as, but not limited to, wireless technologies that can include Li-Fi, RFID, NFC and hybrid tags and an is card (active, passive, hybrid or battery assisted passive tags), a machine-readable code such as a universal product code or OR code that can comprise an array of black and white squares and can store data such as URLs or other information to be read by a camera on an appliance or other interface, bar codes that can be read by RF or bar code devices, thin film labels and applications with sensors and sensors in communication with wireless technologies such as Li-Fi interfaces and RFID, NFC or hybrid tags (“smart tags”), semiconductors, circuits, chip resistors, thin film chip resistors/transistors, memory and networks, electronic temperature and other-sensing labels including real-time sensing capabilities, electronic sensor circuits, plastic semiconductors, chemical sensors such as potentiometric sensors, chemical field-effect transistor sensors, chemiresistors and chemoreceptors. Technologies can further include containers or lids with sensors to monitor temperature, vacuum, humidity, time, container density, acidity levels and gases, chemicals and volatile organic compounds, such as but not limited to, aldehyde, acetic acids, ethylene, sulphur compounds, alcohol CO2, NH3, H2, H2S, O2, N2 and SO2. Sensors can connect to or communicate via wireless or wired methods to transfer data between two or more appliances in communication with network interfaces and networks comprising Li-Fi, Wi-Fi, Bluetooth, internet, satellite and cloud computing technologies and can involve deployed groups of remote servers and software networks to allow centralized data storage and online access to computer services or resources, provide reporting and status messages regarding any process or operation noted herein to an appliance or client device and any other similar technologies described herein and complementary combinations of any technology or others disclosed herein. Technologies can also include artificial intelligence and augmented or virtual reality applications, software or hardware interfaces for use with appliances. All of the technologies and networks disclosed herein “technology” or “technologies” can be combined or function in any complementary order, combination or function with any other technology, network or process disclosed herein.

Product packaging, information and marketing materials as used herein can include any communicative indicia such as icons, abbreviated text, symbols, shapes, colors, forms, or text, that can be digitally, physically, in combination, or by any other means, communicate or connect to, incorporate, read, be written, attach or associate with a product, container or appliance or other product, container or appliance and include URLs, wired or wireless capabilities, cloud, satellite, web or internet data, containers, technologies, consumer goods, electronics, devices, user interfaces or appliances, including user personal data, email and web site addresses, telephone numbers or any other digital, social media or personal address or banking or financial information as described herein. Product information can include technical and specification data, financial, legal and operating information, data and documents such as warranty, technical and operating manuals and technologies, products and services. Product marketing materials can include smart/sensor tags with written data and materials, readers, URLs, price, product, place, promotions, marketing collateral, coupons, promotional materials, recipes, menus, movies, music, sales, visual and auditory materials, discounts, brochures and other printed or digital product information, visual aids used in sales presentations, web content, product data sheets and white papers and any other materials disclosed herein (all of the aforementioned “product packaging” or “packaging” or “marketing”).

Appliances as used herein can include consumer or electronic devices, user interfaces, electronic goods or retail, household and professional appliances including any user interface such as mobile, smartphones, smart speakers, beacons, tablets, lap tops, computers, glasses, watches, wired or wireless wearables, devices and clothing, devices, rings, jewelry and wristbands, printers, cameras, micro-processors and microphones that can include use with an appliance, consumer good, user interface or local or remote computing, cloud, server, network, appliance or device operating system and any other similar electronic device or appliance operating systems including functions and modes that include product ordering, purchasing, payment processing or delivery. Appliances can further include retail display or merchandising cabinets and refrigerated units, microwave oven, oven, induction cook top, UV light systems and irradiation (any of these technologies can be incorporated as a single mode or combination mode in an appliance or interface and can be controlled by an interface or appliance controller, another or external appliance or interface, a product, container or smartphone, smart speaker, beacon, mobile or remote application to function with the other or external appliance functions and modes disclosed herein), stoves, refrigerators, freezers, washer/dryer, vacuum systems, toaster, rice maker, steamer, pasta cooker, crock pot, modular cooking units, portable or handheld devices which can be smart tag reader enabled, RFID light readers, and can combine, connect and communicate with other technologies or appliances in any complementary or compatible combination and in any combinations of the aforementioned appliances as described herein (“appliance” or “host appliance” or “smart control”).

As used herein, a smart/sensor tag is an RFID, NFC or hybrid tag with a sensor portion (“smart/sensor”). A smart/sensor can be configured to detect or measure the presence, level or quantity of a stimulus in a container or ambient area and when detected a sensor portion contacts or interacts with the stimulus thereby changing the smart/sensor resistivity or signal output. The stimulus includes a vapor, a gas, a liquid or a solid. Specific examples of the stimulus include an analyte, a mold, an ethylene, an alkene, an alkyne, an acid, a ketone, an ester, an aldehyde, an alcohol, an ether, a thiol, an ammonia, mono-nitrogen oxide or an amine. The stimulus can be from the food product in the container or a contaminate in the container. Stimuli as used herein is the plural form of stimulus. This signal output change and a product or purchaser identifier can be detected by a reader and sent to a local, remote appliance or network computing device, server or inventory management system to analyze and interpret the signal change. A smart/sensor can connect to, communicate with or affix to a product, container, an appliance or smart/sensor. A smart/sensor can comprise one or multiple sensors to detect different or unique gases or stimuli. A smart/sensor can be configured to detect levels of freshness or spoilage of a product, an ambient gas, element or condition or detect the presence of harmful ambient gases, contaminants, explosives, etc. and provide appliance or client device notifications when a gas level fails, maintains, reaches or exceeds a predetermined profile, signature or comparative database level or combination of levels. A smart/sensor is a technology as disclosed herein.

There exists a need for a method and system to manufacture, prepare, package, display, market and sell a product, a container with a technology for use with an appliance and a method and system to provide consumers with product packaging that includes technologies, communicative indicia, text and smart/sensors to effectively and immediately communicate specific primary, secondary or tertiary benefits such as benefits regarding technologies, usages, interaction and communication, advantages, product availability and complementary usage with appliances and consumer goods to a consumer using minimal surface area on said container or product packaging and a method and system to provide consumer product packaging information regarding appliances and consumer goods to effectively and immediately communicate specific primary, secondary or tertiary advantages or benefits of an appliance or container a technology. Also disclosed herein are embodiments for an appliance and for containers with a technology and methods for the manufacture of said containers.

For example, a combined primary, secondary or tertiary brand logo or name descriptor can include the use of any of the following communicative indicia or text to form a benefits descriptor and association and in any combination hereof or as disclosed herein:
[primary brand logo or name, icon, generic]
[combined description of association or inter-relation, icon]
   [secondary brand logo or name]
      or
   [tertiary brand logo or name]
      or;
[primary brand logo or name, icon]
[combined description of association or inter-relation]
[secondary brand logo or name]
[combined description of association or inter-relation]
[tertiary brand logo or name, icon, generic]
   or;
[primary brand logo or name]
[secondary brand logo or name, generic]
   or
[tertiary brand logo or name, icon]
[combined description of association or inter-relation]

For example, product packaging can include a primary brand logo or name which includes a product in a container with a technology and a secondary or tertiary brand logo or name or icon for any of the following: an appliance, a smartphone, smart speaker, beacon, smart/sensor, user interface, a retail grocery store, food distribution, delivery or service company such as an internet, cloud or product provider or any others disclosed herein. The logo and brand name for an appliance or technology immediately informs a consumer that there are additional non-obvious or unseen benefits associated with a product, container and technology and a logo for a food distribution channel such as a retail grocery store or a cloud service, immediately informs a consumer of same or other similar type product availability and that this information can be located, read, downloaded or accessed in whole, part or additionally provided via a smart/sensor and accessed with a reader enabled appliance. This is applicable to all the disclosure and examples provided herein:
[primary brand logo or name]
[secondary brand logo or name, icon]
   or
[tertiary brand logo or name, generic, icon]

A container, product packaging and marketing materials can include a specification, technical, benefits or advantages descriptor line or section which states, lists or describes a container technology used with said product or container followed by a benefits or advantages section which states, lists or describes the benefits offered by the product or container and how said product or container can be used post purchase with an appliance. Additionally, icons or other communicative indicia and text can be used to represent the container technologies, benefits and advantages. This method allows a consumer to view a product, container or technology and product packaging and be immediately informed of the product's or container's benefits, such as, functionalities, technologies and advantages and informs a consumer which appliances can utilize said product or technologies. Also, by using a standard section or informational format on a product, container, product packaging, marketing material and smart/sensor a consumer can quickly and easily find this information on any product, brand, container, product packaging or smart/sensor to efficiently and quickly obtain product and container technology information or information regarding which brands, products or appliances are capable of using said technologies while being informed regarding said product availability. Examples of container informational benefits sections can include any combination of the technologies disclosed herein, in any combination or order and are not limited hereto:

container technology: smart/sensor, [product name], icon
benefits: track, monitor, order, purchase and payment process
advantages: use with smart refrigerator, smartphone, smart speaker
container specification: smart/sensor
benefits: track and monitor product freshness, auto reorder,
cooking
use with: smart refrigerator, [product logo], icon, smart oven
container technology: smart/sensor, icon

FIG. 5 depicts an embodiment of a product inside a container with a technology and brand logos and names. A sealable container (222) is provided with a cover (221) disposed thereon. The cover can include a one-way valve (220) to allow air to be evacuated from inside said container when a vacuum is applied to it to create a vacuum environment therein. An adhesive, film or plastic material (227) can cover the one-way valve to protect the valve and container contents and can be removed to allow a vacuum environment to be created inside said container. A smart/sensor, or a combination of tags, can communicate or connect with each other, as well as for any of the other embodiments described herein, and can be located individually or together inside or outside said container or appliance interior, to monitor, track and report a level of product freshness, gas levels or contaminants, identify a product or container or the location of a product, a container or an appliance in a retail or home setting. A primary brand logo or name (223) can represent a product. A secondary brand logo or name (224) can represent a technology such as a smart/sensor or a cloud service that provides a subscription service to detect product freshness levels or to order or replace a spoiled product. A brand logo or name descriptor (225) can describe or represent brand logos or names. A tertiary brand logo or name (226) can represent an appliance that can function with said product, container or technology.

For example, as shown in FIGS. 15, 17-19, a smart/sensor can be disposed inside a closed container with a product. A sensor portion can be configured to include single or multiple sensors to detect gases from the product when sealed inside a container. A sensor can also be configured with a sensor to detect a gas or other stimuli that is not produced or emitted by the product. This type of configuration can allow a smart/sensor in a sealed container to detect gas levels produced by a product inside a container so that a handheld device or an appliance reader can detect a change in the resistivity level or signal output of a tag and, as noted herein, said change can be analyzed and interpreted to notify an appliance or user that a product is fresh or spoiled. Additionally, in response to a change a product order can be placed into a virtual shopping basket or an order can be placed or a request to replace the product if spoiled. Further, one of the sensors can be configured to read a gas or other stimuli not associated with a product or spoilage process. For example, a smart/sensor in a sealed container can detect oxygen, or parts of oxygen: for example, in a retail setting indicating that a container has been opened, tampered with or is broken and provide a notice to a user or an appliance or automatically order a replacement product. In another example, a user opens a sealed container and a smart/sensor inside the container detects a gas or stimuli not in the sealed container or associated with the product or the spoilage process, but detects an ambient gas or other stimuli, which can be analyzed and interpreted as an open container and for an order to be placed. The change analysis and interpretation can be configured for a retail or home environment. Any detection or order can provide a user or appliance with a notice, an option to purchase or replace or to place a product into an online or virtual shopping basket and provide a user or appliance confirmation for each process. Additionally, a product delivery can be requested with each action with a confirmed time or date or provide available delivery times/dates for a user to select which includes a delivery confirmation. An order can be made immediately, provide for a delay period such as 24 hours and only allow one purchase per product per a determined time period unless a user indicates otherwise in a profile.

In the following description, for purposes of explanation and not limitation, specific details are set forth for particular networks, communication systems, computers, terminals, appliances, devices, components, techniques, storage devices, data and network protocols, software products and systems, operating systems, development interfaces, hardware, etc. in order to provide a thorough understanding of the present invention which can apply, function and operate to and with any of the appliances, products, technologies and user interfaces disclosed herein to allow appliances, smartphones, augmented or virtual reality applications and software, Artificial Intelligence (AI), image and event recognition, voice activation and recognition software or hardware and interfaces to connect, communicate and interact with a user, user interface and software and hardware programs and interfaces for connected or wireless communication via cloud, internet, satellite and other types of systems and services to operate, interact and communicate with any of the appliances, technologies, interfaces and networks disclosed herein.

It will be apparent to one skilled in the art that the present invention may be practiced in other embodiments that depart from these specific details. Detailed descriptions of well-known networks, computers, digital devices, storage devices, components, appliances, technologies, techniques, data and network protocols, software products and systems, development interfaces, operating systems, and hardware are omitted so as not to obscure the description of the present invention.

The operations described herein can be implemented as executable code stored on a computer or machine readable non-transitory tangible storage medium (e.g., floppy disk, hard disk, ROM, EEPROM, nonvolatile RAM, CD-ROM, etc.) that are completed based on execution of the code by a processor circuit implemented using one or more integrated circuits; the operations described herein also can be implemented as executable logic that is encoded in one or more non-transitory tangible media for execution (e.g., programmable logic arrays or devices, field programmable gate arrays, programmable array logic, application specific integrated circuits, etc.).

FIGS. 6 and 9 describe a product management system 100. The system 100 comprises a plurality of user interface devices 120 and a main server 150 interconnected via a communication network 140. Various networks 140 may be implemented in accordance with embodiments of the invention, including a wired or wireless local area network (LAN) and a wide area network (WAN), wireless personal area network (PAN) and other types of networks. When used in a LAN networking environment, computers may be connected to the LAN through a network interface or adapter. When used in a WAN networking environment, computers typically include a modem or other communication mechanism. Modems can be internal or external, and can be connected to the system bus via the user-input interface, or other appropriate mechanism. Computers can be connected over the internet, an intranet, extranet, ethernet, or any other system that provides communications, such as by the network 140. Some suitable communications protocols may include TCP/IP, UDP, OSI, Ethernet, WAP, IEEE 802.11, Bluetooth, Zigbee, IrDa or any other desired protocol. Furthermore, components of the system may communicate through a combination of wired or wireless paths.

The system 100 can be accessed via any user interface device or appliance 120 that is capable of connecting to the main server 150. A user interface device or appliance 120 comprises a display, and preferably a touch screen display, reader, a video/camera and a microphone for inputting voice/sound. An exemplary user interface device or appliance 120 contains a web browser or similar program, allowing in some embodiments for a secure SSL connection, and able to display HTML and CSS. This includes user interface devices or appliances 120 such as tablets, iPads, Mac OS computers, Windows computers, e-readers, and mobile user devices such as an iPhone, Android, Samsung and Windows Phone. Preferably, the user interface device or appliance 120 is a smart: appliance, phone, speaker or tablet. The user interface devices or appliance 120 can connect to the server 150 via the internet and/or wirelessly, such as through a mobile telephone, cloud or satellite network 140, and/or any other suitable medium. User interface devices or appliances 120 are able to communicate to the main server 150 so that content can be started on one user interface device or appliance 120 and later continued on a separate user interface device or appliance 120. The user interface device or appliance 120 preferably includes an I/O interface that allows a user to interact with the system 100. The I/O interface may include any hardware, software, or combination of hardware and software. For example, a smart watch or any other appliance or interface, can be configured to control the operating system of an appliance or cooking network, such as an oven or microwave as described herein.

The CPU of the user interface device or appliance 120 can be implemented as a conventional microprocessor, application specific integrated circuit (ASIC), digital signal processor (DSP), programmable gate array (PGA), or the like. The CPU executes the instructions that are stored in order to process data. The set of instructions may include various instructions that perform a particular task or tasks, such as those shown in the appended flowchart. Such a set of instructions for performing a particular task may be characterized as a program, software program, software, engine, module, component, mechanism, algorithm or tool. The memory may include random access memory (RAM), ready-only memory (ROM), programmable memory, flash memory, and the like. The memory, include application programs, OS, application data etc. The exemplary computing device 120 can also include a network module connected to an antenna to communicate with rest of the system 100.

The main server 150 described herein can include one or more computer systems directly connected to one another and/or connected over the network 140. Each computer system includes a processor, non-volatile memory, user input and user output mechanisms, a network interface, and executable program code (software) comprising computer executable instructions stored in non-transitory tangible memory that executes to control the operation of the main server 150. Similarly, the processors functional components formed of one or more modules of program code executing on one or more computers. Various commercially available computer systems and operating system software can be used to implement the hardware and software. The components of each server can be co-located or distributed. In addition, all or portions of the same software and/or hardware can be used to implement two or more of the functional servers (or processors) shown. The main server 150 can run any desired operating system, such as Windows, Mac OS X, Solaris or any other server based operating systems. Other embodiments can include different functional components. In addition, the present invention is not limited to a particular environment or main server 150 configuration. Preferably, the main server 150 is a cloud based computer system.

The main server 150 includes a web server and the query processing unit. The web server receives the user requests and sends it to the query processing unit. The query processing unit processes the request and responds back to the user interface device 120 via the web server. The query processing unit fetches data from the database server if additional information is needed for processing the request. The database is stored in the non-volatile memory. The term “database” includes a single database and a plurality of separate databases. The main server 150 can comprise the non-volatile memory or the main server 150 can be in communication with the non-volatile memory storing the database. The database can be stored at different locations.

A computing environment can include a server computer or any other device, appliance or system to provide computing functionality which can include a plurality of computing appliances or devices configured in one or a plurality of server or computer banks. In one embodiment, a computing environment can include a plurality of computing appliances or devices including a hosted or grid computing resource or other distributed computing arrangement. Relevant appliance, product, food, recipe and cooking times, expiration date data, order-purchase-processing data and gas, volatile organic compound and chemical profiles and product freshness and threshold levels and images for the aforementioned respective products can be stored in a data location accessible to the computing environment and can comprise a plurality of data stores. Stored data can be associated with the operation of the various processes, applications and/or appliance functions described herein. Stored data can include appliance profiles such as an oven, microwave, multi-function appliance, appliance cooking network, smart control or smart refrigerator.

Software program modules and data stored in the non-volatile memory of the main server 150 can be arranged in logical collections of related information on a plurality of computer systems having associated non-volatile memories. The software and data can be stored using any data structures known in the art including files, arrays, linked lists, relational database tables and the like.

The server 150 and user interface devices or appliances 120 are programed to perform the methods and processes described herein. For example, a smartphone, appliance or device such as an Apple, Nokia or Samsung can include a smart/sensor reader and use an application to access marketing information and information from said smart/sensors and to manage said content either alphabetically, by store location or product type and a user can add personal notes and other information to said retrieved materials such as comparative pricing information and material, store name and location of said product, time, future sales dates, discounts, product specifications, recipes and ingredients and to read, for example, thin film tags in communication with smart/sensors to view recorded gas or temperature and other sensor information, market and transport time and product processing, storage and transportation history of a product or container, product ordering, purchasing, payment processing and delivery information, etc. Said information can be accessed directly from a smart/sensor or via a product, appliance or device application that can access said information wirelessly via satellite, Li-Fi, cloud or internet communication or connectivity or as described herein and can include tracking, monitoring and reporting product freshness, in containers or appliance compartments and can enable product ordering, purchasing, payment processing, product delivery and any others described herein. A network or appliance can connect or communicate directly or indirectly with a local, remote or network computing device, server or product inventory/purchasing management system or other means to a third party product/service provider such as online or physical retail/wholesale grocery store, product delivery service or online payment service, credit/debit card provider/service or product/service payment, financial or bank provider to allow and facilitate product ordering, purchasing, payment processing or product delivery. For example, any of the appliances, local, remote appliance or network computing devices, servers or product/inventory management systems can contain a purchaser identifier (name, address, biometrics as disclosed herein, financial/banking information, contact information and payment methods as disclosed herein) which can be a purchaser, user, individual, business, corporation, etc., which can added to an order to identify and complete a replacement or purchase order, payment processing or business or home delivery request or order.

Food can comprise data corresponding to a type or class of food or a packaged food or item that can be stored, preserved or cooked inside an appliance, such as an oven, microwave, smart refrigerator, etc. Each food or item can include an identifier such as a smart/sensor tag, bar/QR code, etc., a product image model and a gas signature or freshness threshold level (fresh, ok, consume, almost spoiled, spoiled) with recipes, cooking times and food data based on the food profiles to cool, defrost, cook or warm a single or a plurality of products with an appliance cooking network to synch a set meal time for each appliance.

A smart/sensor, bar/QR code, etc., identifier can provide information identifying a product, a product container, which can include the type(s) of food and cooking times, preparation and treatment such as defrosting, refrigeration, cooking and warming instructions with cooking times, for the one or more products that can be used with one or more wire or wireless connected appliances in a cooking network that can include serving size; quantity, amount or weight of a product or container in each appliance, and a unique appliance identification or identifier and location of the product or container with a product such as use with a single or plurality of appliances such as an oven, microwave, multi-function appliance or smart refrigerator.

As provided herein, a smart/sensor and a camera can each include network interfaces. For example, the smart/sensor and camera can connect and communicate with each other and be in data communication with the computing environment through a network interface. This allows a smart/sensor and a camera to directly communicate with various data stores and applications across the network, cloud, satellite and others. A camera with a network interface can send a food or container image to a cooking or product identification application and a smart/sensor can send a gas reading to a gas signature identification application to identify/detect a product freshness level threshold.

Another embodiment includes an appliance with a smart tag reader and a camera connected to or in communication with satellite, cloud computing or internet network applications to read container smart/sensors and products into an appliance inventory system and record container open date status and product expiration dates which can also be network connected to food distribution and service provider networks as described herein. A consumer using an appliance or secure code access, website or network in communication with an appliance operating and inventory management system can be configured to access, view, review and monitor appliance cooking and status processes or inventoried products and to place product orders via a connected food distribution and service provider network. Containers with technologies can be configured to operate and control product ordering, purchasing, payment processing, delivery and appliance temperature, humidity, venting and other operating settings.

For example, as shown in FIGS. 10 and 20, an appliance inventory management system can comprise a digital list of products with smart/sensors created by a user with an appliance program or app (“app”). The user creates a digital list of basic, shopping or retail or house room products (“list”) and the quantity or number of products to be maintained in an appliance, pantry or retail or house area, such as the living, bed or bathroom (“modules” or “room”). This data list can be entered and stored into an appliance or network by a user, a smart tag reader, a camera, voice, via a network connection or lists can be preloaded in an appliance or via another appliance or a combination as disclosed herein, with said products identified as basic, shopping or room products. The appliance program or app (“app”) can be configured to function in several modes. An app mode can read all smart/sensors within reading range in an area or room and display all the smart/sensor products on a handheld device or appliance display. The app can be configured to display only the smart/sensor products that are not identified or that are absent within a reading range when compared to a stored data list in a handheld device or appliance. The app can operate in a room mode; for example, a user can select a bathroom mode and the app can be configured to read and display only products that are in a bathroom such as products intended to be in a bathroom based on a stored or provided marketing list or products that are missing or absent from a bathroom when compared to a stored list or products that do not belong in a bathroom or can suggest products based on the room and from the stored lists and data bases for the smart/sensor reader, smart/sensors or camera image database. For example, an app mode allows a user to read, list and display products that should ‘not be present’ in a room. For example, a user can select this mode for a living room and the app scans/reads the area to only identify smart/sensor products that do not belong in the room such as identifying a smart/sensor tube of toothpaste thereby informing a user that this product should be located in another room such as bathroom. In this manner a user can quickly walk through the home or a retail environment and quickly identify products that are present or available, identify products that are absent or missing, identify products that do not belong in a room or area and identify products that need to be ordered and to also receive product suggestions for each room or area with each reading or room mode. For example, an app room mode can be configured into other apps or apps can be configured to operate individually or in any sequence with the capability to store search results or send them to other appliances or networks. For example, when an appliance, pantry or room smart/sensor reader or a camera (smart/sensors and readers can be connected to or in communication with a camera and database) cannot identify or read a basic, shopping or room list product in an appliance, pantry or retail/house area that product data can be sent to an appliance or network with a notice or message regarding the product status or said data can be sent directly to a network to automatically place said product into an order basket or place a product order, purchase, payment processing or delivery request for said product(s). The app ordering function can also place an order after a predetermined time period, such as after a 24 hour period. An app can also be connected to or in communication with a camera or image database. For example, if a reader and computing device does not identify a smart/sensor product in an appliance, pantry or retail/house area, and prior to placing an order based on the product not being present, an app can compare the inventory management data stores/bases with camera images, voice and smart/sensor products or databases as well as pending shopping orders or recent purchase orders to verify the same or similar product is not available and can also compare and review virtual shopping baskets. Therefore, when a smart/sensor reader detects an absent or missing product in an appliance, shopping or room list, the smart/sensor data base can query the camera image, voice or text product data base to confirm or verify the presence or absence of a product from one or more rooms prior to sending a notification or placing an order and the camera, voice or text ordering functions can operating similarly with the respective smart/sensor reader and product data bases. In this manner if a smart beacon detects that a box of tissues is absent or missing from a room the app can query the room and data bases to locate a product if it has been moved to another room or location, can detect a similar product placed or stored inside a pantry, can identify a pending order or virtual shopping basket with the product or similar product and provide a user a notice or place an order. An app can also have a mode to only identify and display a digital list of products that have freshness levels or expiration dates within determined times or dates, such as days, weeks or years. The app can also be configured to only identify certain types or groups of products such as fresh foods including meat, fish, dairy or fruit and vegetables or container products. An appliance app can be configured to create, read and display a list of products in an appliance, pantry or retail/house environment in alphabetical order, food or product group, gas freshness level and expiration date, expiration date order, to search for specific recipe products or a product group, type or specific product can be inputted into an app to quickly search for the product. An app can provide a list of products that are present, absent, in the incorrect room or location or can suggest or recommend products based on products identified and stored in appliance, room, camera, voice, text and smart/sensor data bases. For example, a user can walk into their living room, bathroom, pantry or open their refrigerator with a smart control, as explained herein, and the smart/sensor reader or camera can immediately inform the user that a box of tissues, toilet paper, toothpaste or baking soda is absent or missing and to place an order or to replace said absent product with one from another area in the house. The app can also provide the user with information such as how long a product was present in a room or area until the product was detected as absent. The appliance program or app can then either place said product into an online or virtual order basket, and upon reaching a predetermined dollar amount can send the order, or send a product request to order, purchase, payment process or deliver for said products or via a third party product/service provider. Delivery request information can be sent by the order recipient, delivery service or connected network to a user with a schedule of available times to select from and to confirm or a specific delivery time confirmation can be sent. A notice and confirmation for each or any step of the inventory management process can be provided to a user. The aforementioned and all disclosure herein can also apply to product ordering via voice or camera by capturing voice and images and transmitting them to local, remote or network computing devices, servers or product inventory/ordering management systems which can query, connect and communicate with smart/sensors, voice or camera image data storage bases/stores.

### BRIEF DESCRIPTION OF THE DRAWINGS

FIG. 1 illustrates a perspective view of a housing with compartments.

FIG. 2 illustrates a perspective view of an appliance drawer.

FIG. 3 illustrates a perspective view of an appliance with containers.

FIG. 4 illustrates an embodiment of a schematic diagram of a control system.

FIG. 5 illustrates a perishable food or item inside a container with a container technology and brand logos or names.

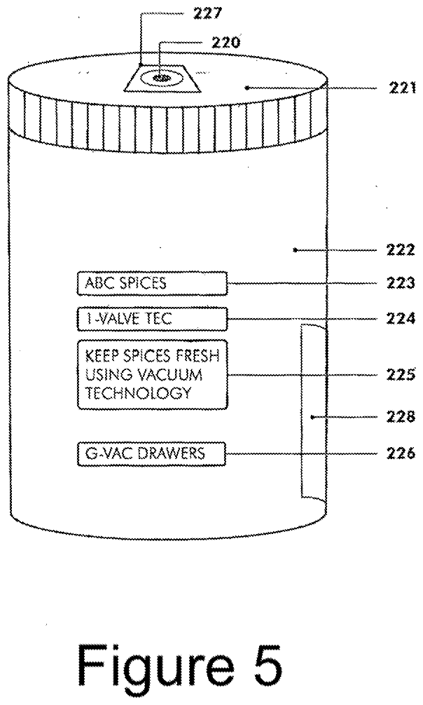

FIG. 6 describes a food or goods management system.

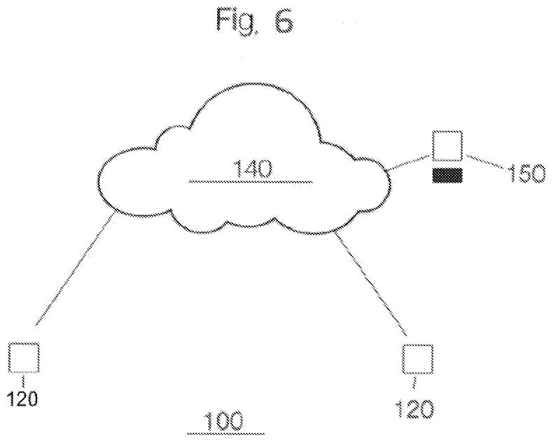

FIG. 7 illustrates a flow chart for an appliance.

FIG. 8 illustrates a flow chart for an appliance.

FIG. 9 illustrates a flow chart for an appliance.

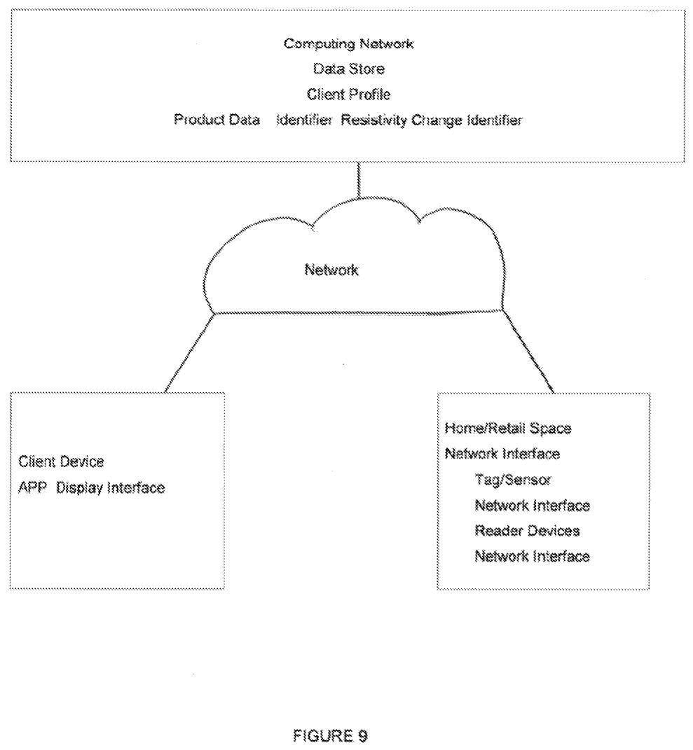

FIG. 10 illustrates a flow chart for an appliance.

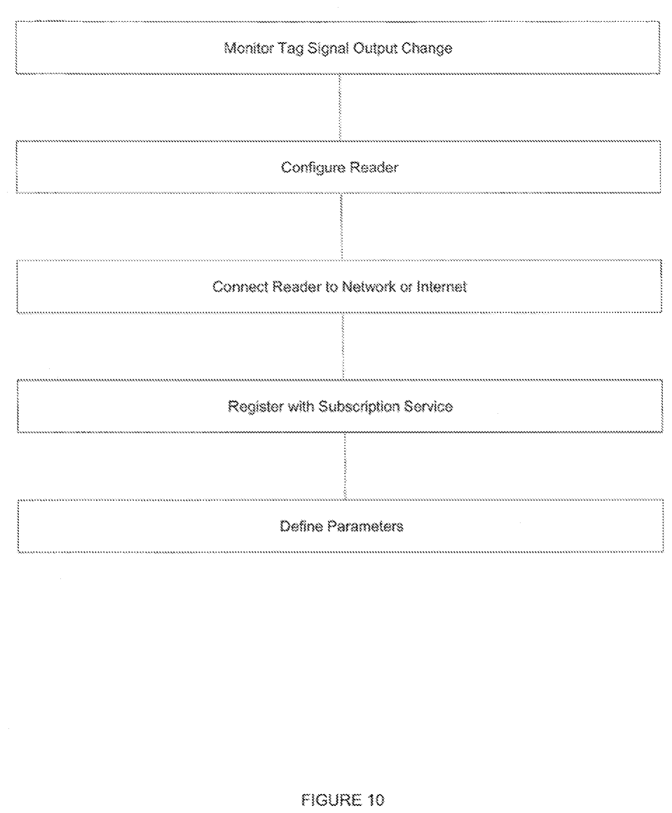

FIG. 11 illustrates a side view of a host appliance with an appliance.

FIG. 12 illustrates a side view of an appliance with an induction plate.

FIG. 13 illustrates an appliance input/output port or connection and interface with cable for a smart control or host appliance.

FIG. 14 illustrates a front view of a smart control in a base.

FIG. 15 illustrates a block diagram for an appliance or network process.

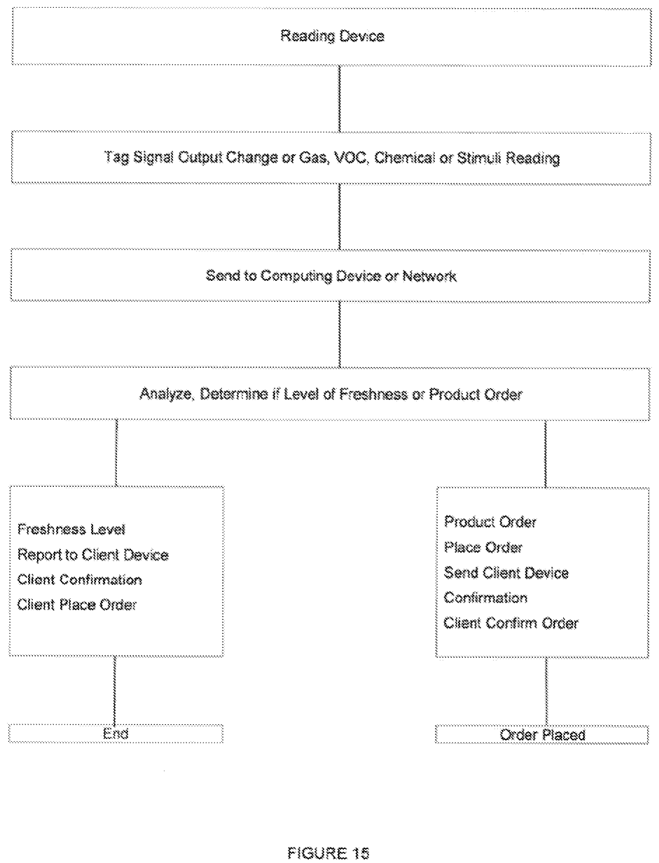

FIG. 16 illustrates a block diagram for an appliance or network process.

FIG. 17 illustrates a block diagram for an appliance or network process.

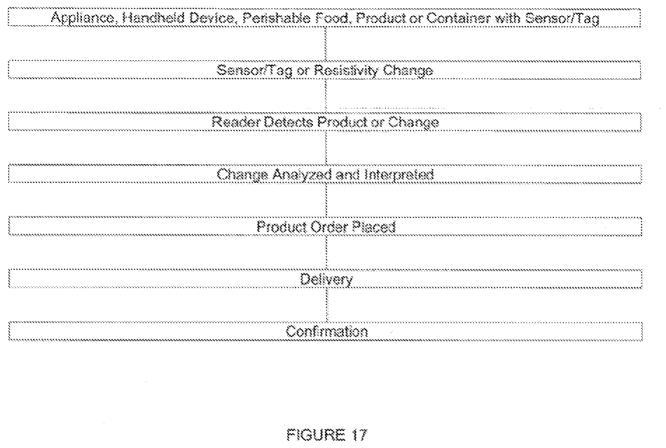

FIG. 18 illustrates a block diagram for an appliance or network process.

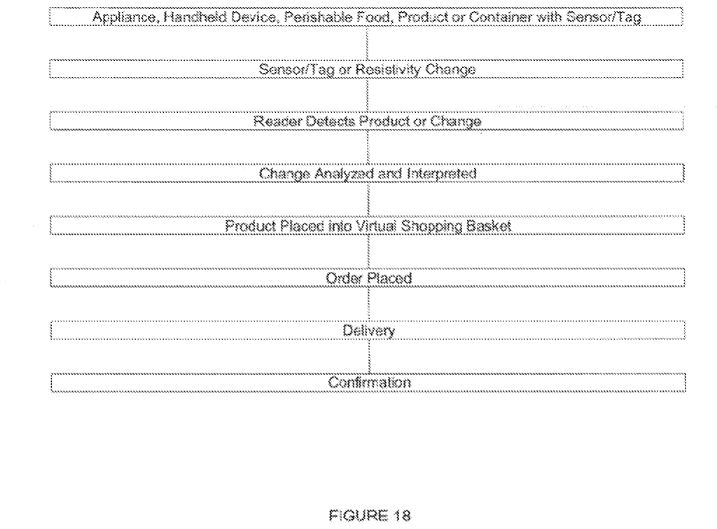

FIG. 19 illustrates a block diagram for an appliance or network process.

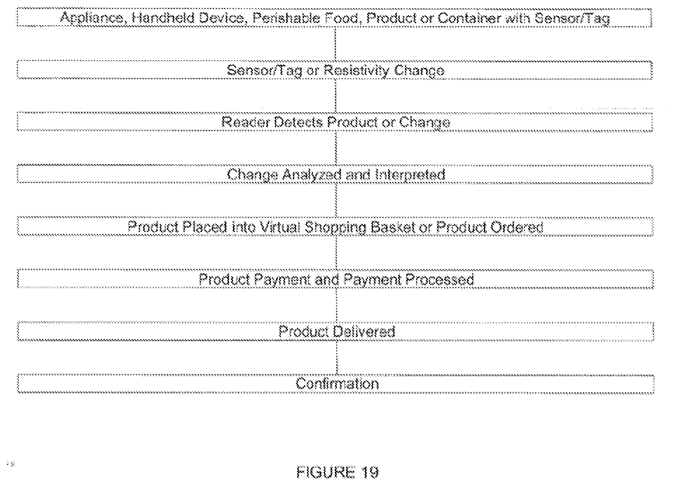

FIG. 20 illustrates a block diagram for an appliance or network process.

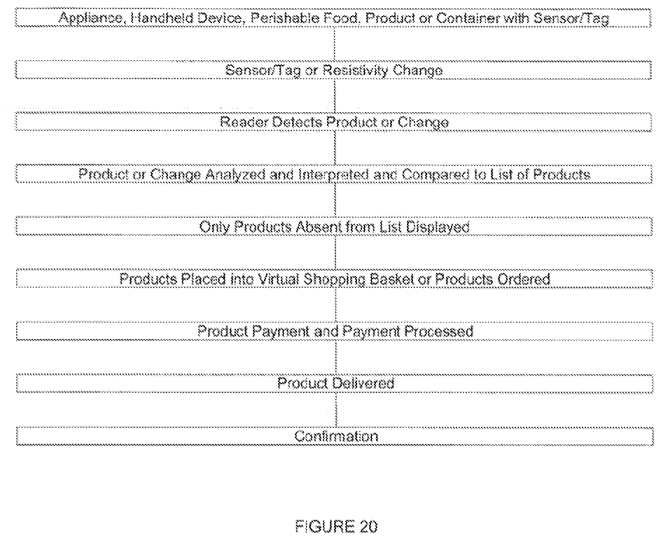

### DETAILED DESCRIPTION OF THE INVENTION

The invention will now be described with reference to the attached non-limiting

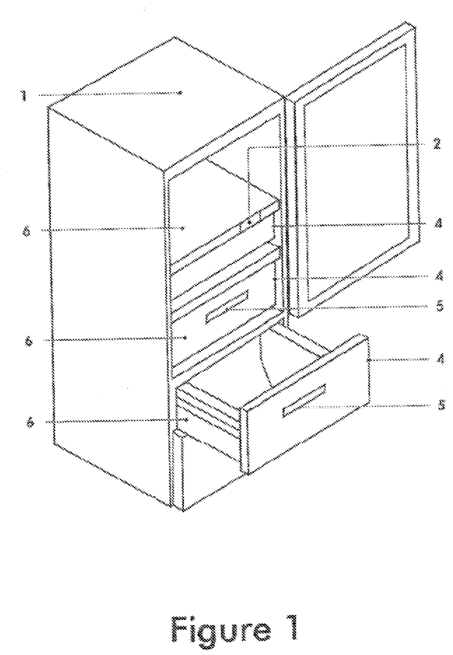

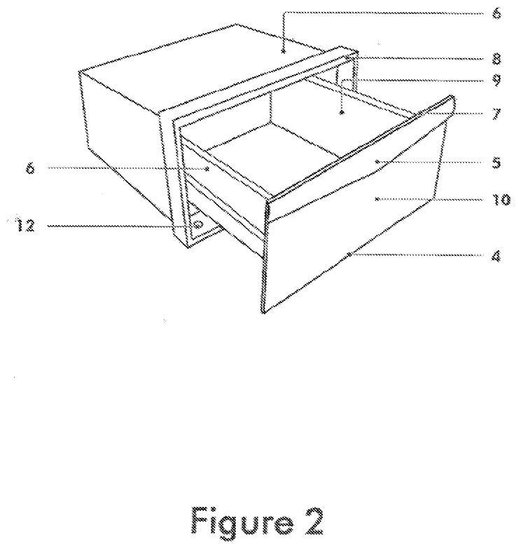

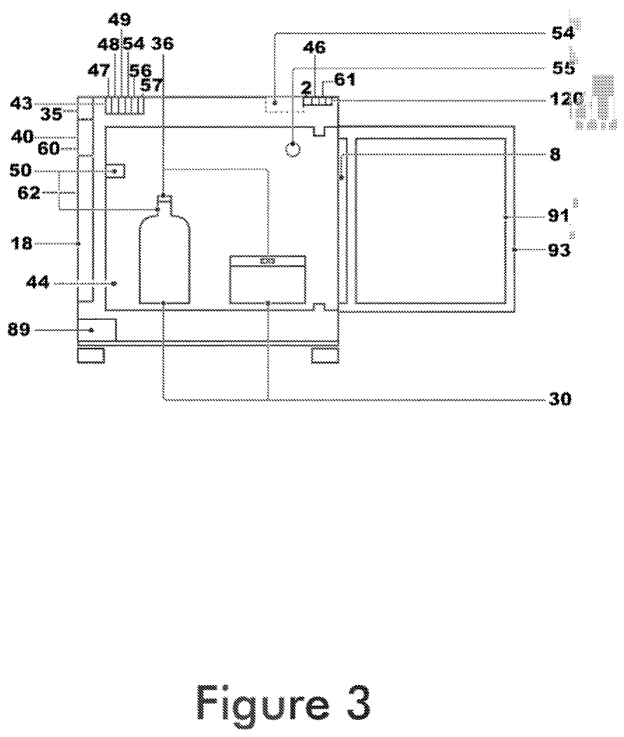

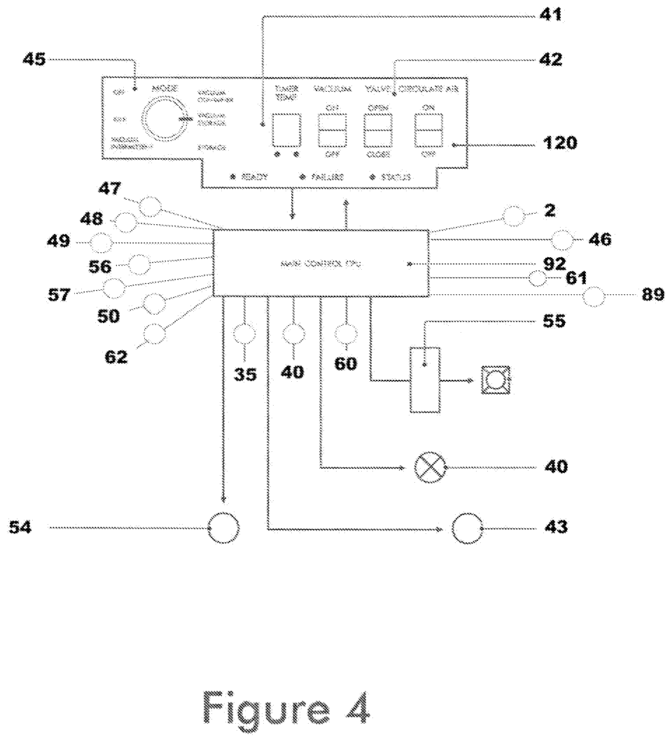

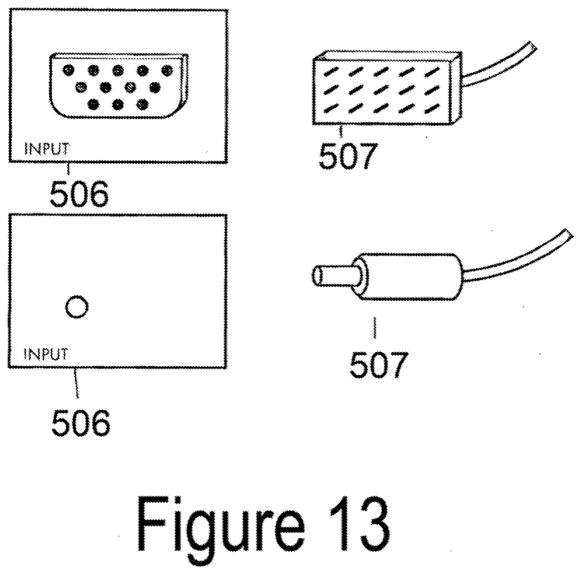

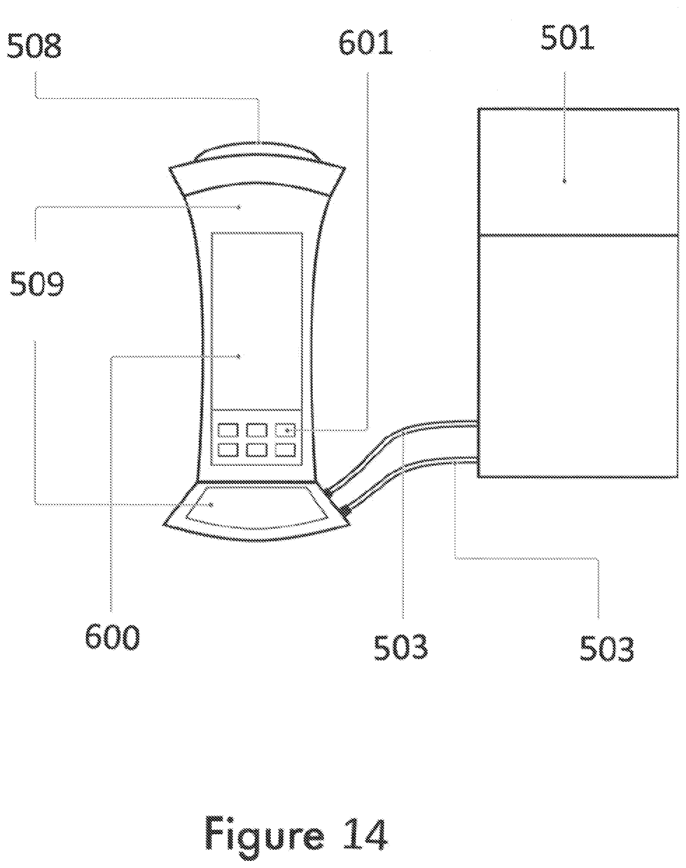

Figures. As shown in FIGS. 1-3, preferred embodiments include an appliance, multi-function appliance or appliance control system (“appliance”, “host appliance”, “smart control”) with an appliance comprising any combination of the following functionalities, modes, elements and controls, but not limited thereto: an appliance compartment constructed to function as or with a vacuum system, an oven, a microwave oven, an induction cooktop (“induction plate”), an electrical cooking heating element, a temperature controlled compartment or housing with a temperature means that can include conventional refrigeration cooling and heating systems, a Peltier device or solid state cooling and heating (condenser and condenser free systems)(which can be located inside or outside an appliance compartment), a vacuum bag sealer with a heat bar sealer situated inside a compartment, a UV light source system, a handheld or manual container vacuum system, a handheld or manual UV light system, a humidity and venting system, as well as, the other previously disclosed wireless capabilities, functions and modes, that can be smart/sensor reader enabled and connect and communicate with an appliance control or operating system and can be controlled wirelessly via a smartphone, voice, AI, an appliance or an external appliance. In one embodiment, an appliance can comprise any of the following such as, a housing (1) in which at least one sealable modular compartment (6) can be disposed with at least one or more containers disposed therein. A vacuum pump can connect and communicate with the interior of the housing to create vacuum, pressure, venting and other environments inside said compartment and inside one-way valve or seal containers disposed therein. As shown in FIG. 4, an appliance control system (2) can include a display panel, microprocessor (CPU), memory device, network interface and software and hardware for a network enabled appliance including a wireless device connected to a microcontroller to communicate with cloud, internet or satellite networks, a smart control, other appliances, containers and to control and operate each system. Furthermore, a smart control (as shown in one embodiment in FIG. 14), a smart power-strip and an appliance can be constructed to incorporate input/output ports (506), as shown in one embodiment in FIG. 13, an interface or a micro-controller to allow a user to connect a smart control, a smart power-strip and an appliance together to communicate to allow software, an interface and micro-controllers to connect to communicate with hardware devices and with respective control and operating systems. As used herein an input/output port (507) can include any system, including cable or wireless, to couple a host appliance, a smart control or another appliance together to allow the creation of a network of cooking appliances to communicate and share information, data, electricity, wireless connectivity via cloud, internet, satellite, Bluetooth and control by a host appliance, smart control or appliance in the network. The coupling device can include an input/output port, slidable connectors, click or snap connectors, etc. The appliance network can also operate with appliances to connect to the network to function as a host appliance or as an appliance or a recipient of network data, connectivity, and electricity, among others.

An appliance control and operating system can selectively or automatically activate and deactivate a vacuum pump and place said pump in either a vacuum, pressure or vent mode. A pump can connect to the interior of a compartment and communicate with a control system, smart/sensors and release valves connected to and in communication with said compartment. In differing embodiments, operating systems can be physically located inside or outside, or a combination, of an appliance compartment. Furthermore, said compartment cavities, spaces and apertures as disclosed herein can be constructed and designed to support a combination of microwave oven (RF), induction plate (505), a food heating element, oven, UV light source and environments such as vacuum, humidity, outgassing, venting, and temperature modes or functions and others disclosed herein (“environments”).

Another embodiment provides an apparatus housing (18) and control and operating systems to create, monitor, regulate, maintain and release compartment or container environments and can connect to and communicate with a smart control, appliance operating systems, smart/sensors and a compartment. Operating systems, as described herein, can include product inventory, ordering, purchasing and payment processing systems and an appliance, smart control or smart power-strip function and mode to create a network of communicating appliances which can include smart, dumb and cable connected appliances, an appliance cooking network using cooking synching algorithms, software and hardware interface system for local or remote data for a smart control and appliances which can include voice activation and interaction controls, an AI connected camera with facial, product and event recognition interface, hardware and software for local or remote data including AI software and connectivity via internet, cloud, satellite, etc., pump(s) (89), smart/sensors which can connect and communicate with products, containers and appliances (50) (which can include humidity, gas, temperature, tactile, time, pressure and vacuum, and as described herein), valves (90), system status indicators, controls and reporting with notifications and temperature (40) and humidity (35) control devices, (41) single or multiple cameras (still and video) positioned or situated inside a housing, on a compartment or an appliance door to monitor a compartment interior, contents and record user, product and appliance event and transaction activities (42), ultraviolet light emitting diode (UV light), (43) microwave oven, induction top, an oven electrical heating device for cooking and heating functions, smart/sensor tags and reader systems with associated software and hardware programs and functions (operating system(s), systems and components) and a scanner to read bar and QR codes on containers for food information, preparation and cooking times. Appliances and a smart control, and respective control and operating systems, can connect and communicate with each other using compatible control and operating systems, programs and software. A temperature system (40) can create a range of temperature inside a housing (18), a compartment (6) and a container (30) disposed therein and can connect to a control system (2) and to individual or selected system components. A humidity system (35) can create a range of humidity inside a compartment and a container disposed therein and can connect to a control system and to individual or selected system components. Single or multiple UV lights (42) can be positioned inside a compartment such as on a wall, top or bottom of said compartment with each configured to emit light having a peak wave length in the ultraviolet range to reduce or eliminate the growth of microbes in said compartment and on food, containers or items placed therein.

A microwave oven function can include microwave oven functions, well known in the art, such as power levels, defrost and cooking times (43), among others, to defrost or cook items inside said compartment and can include a camera (infrared) (41) to identify, monitor and interact with the cooking process, products, contents and activity of a compartment and to connect and communicate with a control and operating system and with smart/sensors during the refrigeration or cooking process. In one embodiment, a compartment housing can include, among other elements, a waveguide (45), cooling fan (46), magnetron (47), high voltage diode (48), high-voltage capacitor and a high-voltage transformer (49) or it can include a solid state microwave system incorporating single or multiple antennas or single or multiple RF sources connected to appliance control and operating systems, interfaces and software each with its own port into a compartment or cavity such that relative phase between the sources can be altered to shift nodes and anti-nodes and can further be connected to an appliance control and operating system incorporating cooking and preservation algorithms, software and feedback processes to control cooking and related functions including other related elements all well known in the art. Additionally, a compartment can also include a vacuum hose attachment (50) in communication with and in connection with a vacuum pump and system under separate switch or control or under control of an appliance control and operating system to create vacuum environments inside one-way valve containers and bags when the aperture of the hose is placed over a one-way valve, seal or other vacuum sealing mechanism on said container or bag and said vacuum system is activated. The vacuum hose attachment can be removably incorporated into a housing or compartment. An assembly can include and comprise one or more external vacuum hose input ports connected to and in communication with a vacuum pump in said appliance compartment or housing to apply a vacuum to containers or bags. Said vacuum hose system can be manually activated via a switch or via an appliance control and operating system.

Furthermore, a handheld UV light (51) and container vacuum system can be removably incorporated into a housing or compartment as described above and can be activated or controlled manually by a switch or controlled via an appliance control and operating system. A handheld UV light and handheld vacuum system can connect to and communicate with an appliance control and operating system or can be a wireless unit with a recharger or system incorporated into an appliance housing or compartment.

A compartment storage cavity or space can include a mechanized turntable (52) to rotate with items placed thereon when the microwave oven or UV light treatment functions are activated and can connect and communicate with an appliance control and operating system. A turntable can be made of any materials consistent with the use and need of a microwave oven or the use with a UV light treatment source.

Furthermore, if said appliance is a combination vacuum sealing and microwave oven compartment, when the vacuum sealing function is selected a user can manually seal the holes/apertures inside the compartment cavity that allow delivery of the microwave energy into the compartment cavity and venting with a cover or plate to form a hermetic or vacuum sealed compartment. The cover or plate (53) can be a friction cover such as a concave/convex type mechanism. This function can also be electronic, mechanical and controlled via a control and operating system such that when a vacuum function is selected via a control system an mechanically controlled cover or plate can automatically seal the holes/apertures in a compartment with covers. Likewise, when a microwave function is selected, the covers or plates can manually be removed, slide or move to an open position or can be automatically opened using an electronic device or mechanism via the control system connected to a mechanism to allow the holes/apertures to move to an open position to allow microwave energy to pass into the compartment cavity and to vent the compartment. Said covers or plates can be hinged, threaded, slide or push to seal or can comprise off-set holes/apertures such that when moved the holes/apertures of two overlapping plates align to form an open position and when moved to a close position one of the overlapping plates close the holes/apertures and seal said compartment. A venting or duct system can be utilized that can electrically open or close to allow air to enter or leave a chamber or compartment and be controlled as described herein. Furthermore, a compartment venting system can also be activated to function with a microwave function. The appliance system can be programmed to function with a default to the microwave or other selected mode or function. An appliance can include a control and operating system as described herein to communicate with and operate a microwave oven function and the operation of other systems disclosed herein. A compartment can comprise any combination of the aforementioned compatible control and operating systems which can function individually or in combination or sequence with other control and operating systems.

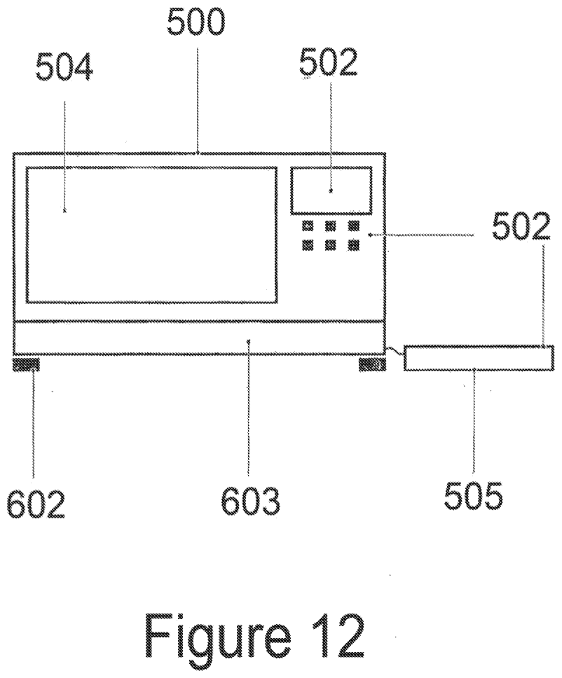

As shown in FIG. 12, an appliance can also incorporate an induction plate (505) into a compartment providing individual operating controls incorporated into the induction plate, appliance induction control and operating systems or a combination of controls. An induction plate can be incorporated into the base or bottom of an appliance to create a fixed and sealed appliance compartment component or the induction plate can be removable and can be constructed to fit into a cavity or compartment recess designed to hold the plate. The plate can electrically connect into an appliance compartment. In this manner, a user can open an appliance door placing suitable cookware into the appliance and activate the induction plate with controls on the plate or via the appliance control and operating system. In another example, a user can place suitable cookware into the appliance and close the appliance door. The user can activate the induction plate, as noted, to operate the appliance in a closed position to function as an induction oven and can also activate a heating element which can be placed inside a compartment on the top of the appliance cavity to use individually or in combination with the induction plate. The heating element can also be operated individually, via the appliance control and operating system or in combination with other operating systems and include operating functions, temperature settings and auto shut off, among others. A user can operate the induction plate mode or function and simultaneously operate a venting mode and a microwave mode.

In another embodiment, an induction plate can be incorporated into the top exterior cover of an appliance, as with the interior placement or the induction plate can be constructed to fit under an appliance housing to form and be part of the appliance housing but separate from the compartment cavity. An induction plate housing can also be a stand-alone housing and separate from the appliance housing to hold the induction plate or can be constructed as part of an appliance or attached to the appliance housing and can be affixed on top of the appliance housing. Further, an appliance can be constructed to be placed on top of an induction plate without the use of an induction plate housing so that the induction plate can be moved from under the appliance for use and then moved back underneath the housing to store. An induction plate constructed into an appliance induction housing allows a user to slide the induction plate out of the housing when needed to use the plate to cook an item and can control the plate as discussed. When a user is finished, the induction plate is simply cleaned and pushed back into the appliance housing. The induction plate can be either fixed or removably disposed in this configuration with the appliance housing. The plate can fit into a housing situated or positioned behind the appliance in a vertical type arrangement, where the plate is vertically pulled out to the side and then the plate is lowered down to sit on a countertop. This embodiment can also incorporate the other discussed embodiments herein. In another embodiment the induction plate can be constructed to fit inside an induction plate housing which is separate from an appliance housing to slide the induction plate into and out of the housing for use and storage. An appliance can be placed on top of the induction plate housing, which holds the induction plate, in order to provide more countertop space.

The induction plate can be designed to electrically connect to the appliance housing, appliance control and operating systems or can connect directly to a wall socket. An appliance, induction plate and induction plate housing can incorporate appropriate electrical sockets to interconnect and can provide electrical current to operate both the appliance and induction plate. In another embodiment, an appliance can be designed with legs (602) to provide sufficient space underneath the appliance so that an induction plate can be moved and stored under the appliance. In another embodiment the induction plate can be affixed to the side of an appliance external housing wall, for example, by means of a locking, hydraulic, spring device, hinge or similar mechanisms and combinations, so that a user can unlock or move the induction plate away from the appliance external wall and place the induction plate onto a countertop so that the induction plate lies flat on a countertop for use and can then be returned to its original position to store the induction plate. The individual, appliance and combined controls for the induction plate can include power, function, memory, recipes, temperature settings, oven, plate, auto off, network interface, wireless connectivity and any others mentioned herein. The appliance and induction plate control and operating systems can connect and communicate with each other wirelessly or by a cable connection and the induction plate can function using the appliance operating system, its own operating system or the operating system of a smart control or appliance. An induction plate can be removably disposed to use for camping and other cooking locations and then returned and stored with or in appliance housing.

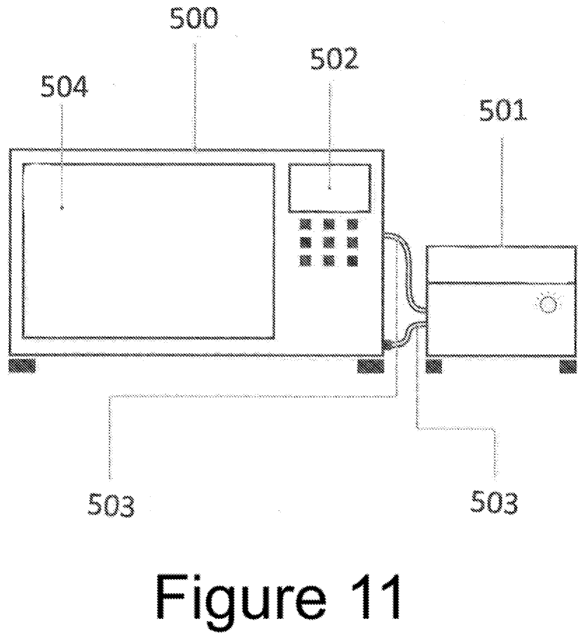

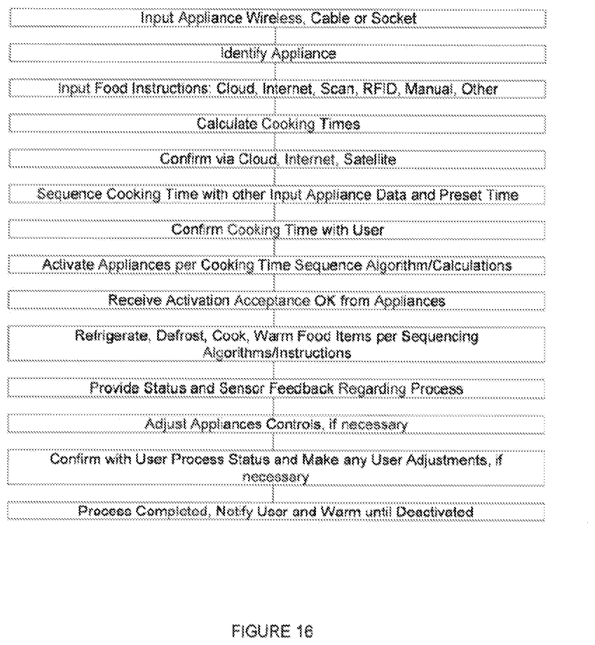

As shown in FIGS. 11 and 16, a smart control (508) or host appliance (500) can incorporate an interface and system to create a network of smart, dumb and cable connected appliances which can connect and communicate with multiple cooking appliances in a network to synch the appliance cooking times to prepare a meal for a set time using a smart control, host appliance or appliance. A smart control and appliance control and operating system can include a network interface, programmable cooking and timing software, hardware and algorithms to wirelessly connect and communicate with appliances via Wi-Fi, cloud, satellite or internet and to connect and communicate with appliances that are not wirelessly enabled by connecting these appliances to a smart control or smart control base or a host appliance using single or multiple electrical sockets situated on, connected to or in communication with a smart control or host appliance which can each control, activate and deactivate an appliance via electrical socket. Each socket can be controlled and operated by an interface, a control and operating system of a smart control with a base, host or appliance. In this manner, a smart control, host or appliance can simultaneously sequence multiple network cooking appliances with food items to complete a cooking process at a predetermined time and to control all the wireless, socket and cable connected appliances via one smart control, host or appliance. The appliance electrical sockets can directly connect to a smart control or a smart control base under control of the smart control, host or external appliance (501). Further, an appliance electrical socket can connect to an electrical smart power-strip type device which can connect to the smart control or base, host or external appliance. In another embodiment an external appliance can connect and communicate with control and operating systems via a single or multiple input/output cable that can connect to and communicate with a smart control or base, host, smart power-strip or appliance input/output port cable connection and interface and each respective control and operating system and vice versa. In another embodiment, a smart power-strip, host or appliance can connect and communicate to a smart control and control and operating system via an input/output cable connecting the respective control and operating systems or via electrical sockets which can be controlled by the smart control and operating system and software. A smart control operating system can function like a host appliance operating system; however, a smart control is a stand-alone control device or appliance similar to a smartphone that can monitor, control and regulate the operating systems of a network comprising a host, smart or dumb appliance. Dumb appliances can be appliances that do not have wireless connection capabilities or enablement and can connect to an appliance network via a host, smart control or smart power-strip cable input/output port, interface or microcontroller or multiple port system or one or more controlled electrical socket connections.

A smart control (508) can function as a countertop, refrigerator or handheld or appliance device to provide cooking instructions; monitor and control an appliance cooking network; function as a household or room security platform or device by connecting to a camera doorbell system or other home security camera systems and monitor with its security camera system; a camera with a motion detector, GPS, to detect and report levels of gas or ambient contaminants; check product inventory and place product orders, purchases and home delivery, using a smart speaker (509) and can include the control and operating systems of a smartphone or host appliance such as wireless enablement and communication, CPU, memory, control, smart/sensors and smart/sensor reader, camera or video, display panel (600), input/output and programmable capabilities (601), AI, augmented and virtual reality food, preparation, recipe or cooking capabilities with software and hardware to connect and communicate wirelessly, via electrical socket or input/output ports, interface or microcontroller with cable (503) to each host, appliance or smart power-strip control and operating system connected to the smart control. A smart control can be placed into a base which can support and recharge the smart control. The smart control can also create a connection with the base via connectors situated at the bottom or base of the smart control to recharge and to connect the smart control operating system with the base and any appliances connected to the base to control them. Furthermore, a smart control can be placed into a refrigerator. A smart control base can also be placed into a refrigerator and can be connected by a flat, electrical cable that can connect to an electrical source outside of the refrigerator, for example into a wall unit. In this manner, a smart control can be placed inside a refrigerator to track and monitor products, order products, etc., as previously discussed herein. A smart control can also operate with batteries. A smart control can be placed inside an appliance, pantry or house area to monitor products and activity and to order items or can be removed from an appliance such as a refrigerator to read and order products from areas such as the pantry or other house areas. The smart control can also be placed onto a kitchen counter in a base to control the cooking process of a network of appliances as disclosed herein. The smart control can also connect and communicate with online cooking sites or recipes to provide step by step cooking preparation and instructions to prepare a meal and to order products to make specific recipes or suggest recipes based upon existing product inventory and smart/sensor freshness information.

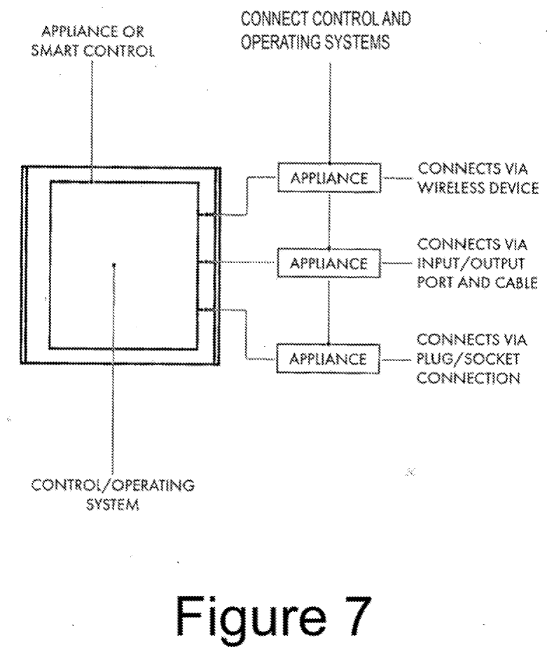

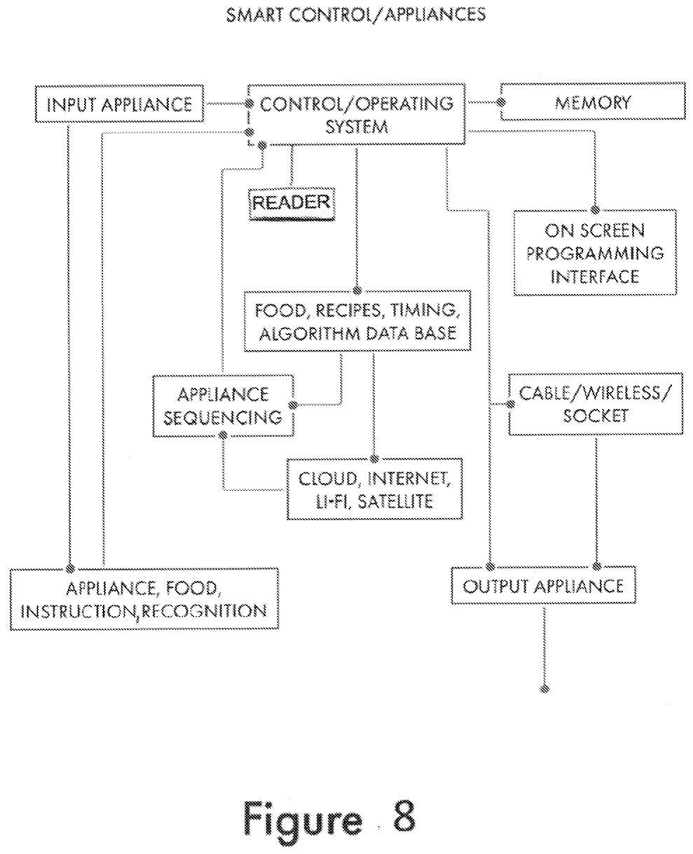

A smart control, host and appliance can use compatible software, hardware and communication systems to connect and operate. A smart control or appliance can control and operate another appliance via cable to activate or deactivate an appliance control and operating system as with the electrical sockets. For example, a rice cooker and a vegetable steamer can be connected to a host or appliance via electrical socket and an input/output port with cables connecting the respective control and operating systems to connect and communicate with or between a host, appliance or smart power-strip to connect and to communicate between the host appliance control and operating system, an appliance or smart power-strip control and operating system. The cooking times, recipe, serving portions, weights, product type and other relevant cooking information for each item and appliance can be network accessed, programmed, read or scanned from smart/sensors or bar/QR codes or data via the internet, cloud, satellite or other, into a smart control or appliance control and operating system or entered into the appliance control and operating system for each connected appliance input/output port with cable or electrical socket for each appliance and host appliance. The host appliance control and operating system and software can determine, control and regulate the network of appliances and calculate and provide the required refrigeration, defrost, cooking and warming times and sequences for each appliance in the network to complete the appliance network cooking cycle at a determined time. (See FIGS. 7-8 and 16) For example, a user can prepare a meal by placing a chicken or fish item into an appliance compartment. The food item can be frozen or sealed in one-way valve or seal enclosed cooking container. The appliance can be programmed to automatically or selectively calculate the required cooking time and sequence for the food item with each network appliance to complete the cooking process for a meal at a determined time period. The system also allows for appliance ambient and function feedback to optimize the cooking and sequencing for the appliance network. The appliance can select to defrost the food item using an RF microwave function or can select to preserve and cool the item until it is ready to cook the item using a solid state cooling function or compartment venting function. The appliance can then cook the food item using the RF microwave function while also calculating or using the provided cooking times to sequence, synch and activate the other network cooking appliances so that the rice and steamed vegetables are cooked and ready to eat at the same time the chicken is finished. After the food items are cooked the smart control or host appliance can control and regulated each appliance to warm the food items until a user is ready to eat and deactivates the system. The food cooking calculation, sequencing and timing can be based on the smart control, host or appliance control and operating systems. This allows a user to effortlessly plan and prepare a meal and have it prepared at the same time. Furthermore, an appliance network program can be activated, controlled, delayed or interrupted using a smartphone or remote application which can automatically make the necessary cooking adjustments to the smart control, host or appliance by connecting and communicating with each appliance control and operating system. In this way, a user only has to communicate with a smart control or host appliance to control and communicate with the network of connected appliances to plan and prepare a meal. Furthermore, all the appliances in the network do not need to be Bluetooth, Wi-Fi or internet enabled. CPG-food containers can incorporate smart/sensors and bar/QR codes to provide recipe, cooking and sequence times which can be read or scanned into a smart control or appliance via a reader or scanner for a smart control or appliance for the network appliances that are connected to and in communication with a smart control or host appliance. CPG-food containers with smart/sensors and bar/QR codes can provide information and data to control a smart control or appliance control and operating system, functions and modes as noted.

Further, a smart control, host appliance or an appliance can have a unique telephone number to allow a user to remotely access to provide voice commands and to view and monitor a location the smart control is placed such as inside an appliance, in the kitchen or other. Voice commands can also be used via internet protocol or other cable, cloud, Skype or wireless protocols as described herein. Furthermore appliance control orders or commands can be provided using smartphone applications such as text, WhatsApp, email, scanning bar/QR codes or other product identifiers described herein. As noted, a smart control can have a rechargeable base so that the smart control can be docked into a charger and used on a kitchen countertop or inside a refrigerator which can then be removed to use in other locations similar to a smartphone. The smart control can also be used to control a refrigerator either with wireless connection and communication or via connectors at the base of the smart control which when placed inside an smart control base inside and connected to an appliance operating system connects the smart control to the appliance operating system. In this manner a smart control can operate and control the functions of a refrigerator and can be removed for other uses as described herein.

An appliance, in a host appliance or smart control mode, can connect and communicate with a host or appliance control, software and operating systems wirelessly, via input/output ports and interface with a connecting cable or via electrical sockets. A host appliance or smart control can create a unidirectional, bidirectional or multidirectional relationship between and among a smart control, smart power-strip, host and an appliance. Further, a host appliance, smart power-strip or smart control can communicate with smart appliances, dumb appliances that incorporate input/output port interfaces and cable enabled connections between and among appliance control and operating systems and with appliances that can connect and communicate via an electrical socket. As noted, these relationships can be created using a wireless communication or system where each appliance can be a smart appliance. In another embodiment, a smart control or host appliance can be the only smart appliance that connects and communicates with external appliances. The external appliances can connect and communicate via dedicated electrical sockets on or in communication with the smart control or host appliance or can connect directly to an electrical outlet source. Further, the external appliance control or operating system can connect via an input/output cable to the smart control and host appliance control or operating system to communicate and vice versa. Each smart control, host or external appliance can have a unique input/output cable connection for a programmed appliance, type of appliance or product or a general input/output cable connection can be read and identified by the smart control or host appliance to connect and communicate with each respective external appliance that is connected to the smart control or host appliance.

In another embodiment, an electrical smart power-strip type device can connect to a wall or power source. The smart power-strip can be wirelessly enabled to communicate with a smart control or host appliance. An external appliance can electrically connect via socket or via an input/output cable to connect the external appliance control and operating system to connect and communicate wirelessly with a smart control or host appliance, as disclosed herein. The smart power-strip can also be used to connect and communicate directly with a smart control or host appliance. Additionally, external appliances can also connect to other external appliances and to a smart control or host appliance via an input/output cable. A smart control or host appliance can be configured or include a function or mode to only operate an external appliance. For example, a user plans a meal with rice and vegetables. The user retrieves a rice cooker from a cabinet and connects the electrical cord into a wall socket or into a general or defined electrical socket on or in communication with the smart control or host appliance. This electrical cord can also include an input/output cable connecting the external appliance control and operating systems to a smart control or host appliance. The user then connects the input/output cable connecting an external appliance control and operating system to the smart control or host appliance control and operating systems via a dedicated or general input/output connection. The user then connects a vegetable steamer in the same manner. The user scans, reads or inputs the necessary product, container, time, serving portion, weight, etc. and sequence information for each connected external appliance into the smart control or host appliance control and operating system via the input, scanner or reader connected and in communication with the smart control or host appliance or via another appliance and sends it to an appliance. Or if the appliance is a reader enabled microwave or oven, a cooking container with a smart/sensor can be placed directly into the appliance with the cooling and cooking data automatically entered. A camera can also determine the product type and above information to set the temperatures for each cooking step. Additionally, a food scale can be connected to a smart control or host appliance via an input/output connection or wirelessly connected to input said data into an appliance operating system and software to determine cooking and sequence times. The smart control or host appliance software and control and operating systems complete the sequenced cooking process for each connected appliance. A user can then add a piece of fish into the microwave or add a pot of water onto an appliance induction plate and place a sous vide cooker into the pot and connect it as noted, to cook a steak, input said information into the smart control or host appliance and the smart control or host appliance can make the necessary cooking, timing and sequence adjustments for the host and external appliances. Furthermore, a user can use voice, AI or a smartphone or smartphone application to change the meal time, cancel or add one or more food items via the control and operating systems of a smart control or host appliance which in turn can make all the necessary cooking, timing and sequence adjustments and send cooking status messages and provide camera images and monitoring of the products in and of said appliance.

As previously described herein, an oven or a microwave can include a refrigeration system to work in combination with the heating elements to prepare and cook a food item which can include a condenser or a solid state cooling system.

For example, the above disclosed microwave cooking process can be used with a microwave oven, oven or combination appliance with a cooling and cooking element or function. An appliance can include operating systems as described herein for an oven with controls for temperature and heat and fans and to control respective air ducts (open/close) including a frame comprising a refrigeration-cooking compartment with a door. Single or multiple heating elements can be disposed inside the refrigeration-cooking compartment to provide heat to the refrigeration-cooking compartment to defrost, cook or warm food items. A temperature cooling system can be disposed inside the housing or compartment to cool and preserve a food item until it is ready to defrost or cook. Further, an inlet duct can extend between the refrigeration-cooking compartment and a cooling element or module situated inside the compartment or housing. An outlet duct in communication with the refrigeration-cooking compartment can open and close to allow the compartment to be refrigerated or to vent the compartment. A refrigeration system can comprise a compressor, condenser or evaporator or a solid state cooling system which can include a fan or venting system such that when the refrigeration system is connected and in communication with the refrigeration-cooking compartment a refrigerated air path can be created between the cooling system or module and the refrigeration-cooking compartment. The refrigerated-cooking oven and microwave can each be smart appliances with camera, voice and wireless interface controls such as a smart watch, phone or other and with wireless connectivity and cable network capability as described herein.

Furthermore, an oven and warming drawer combination can include a warming drawer which can be a multi-function drawer to warm products or create vacuum environments with other functions as described herein. The vacuum-warming drawer can be constructed into the frame of a kitchen cabinet or frame or housing of an oven appliance or can be a separate appliance. Further, the warming drawer can incorporate the heating coils or solid state heating system of an oven which can work in combination with the solid state cooling system of a refrigerated-cooking oven. All combination appliances disclosed herein can incorporate independent, combined or remote operating systems and further the power control circuitry can also be independent or combined. A vacuum-warming drawer can be located below an oven and supported therewith, can be placed above an oven and be supported therewith and can further be a smart appliance or connected with the oven for smart connectivity. Operating controls for the drawer can be combined with an oven or be separate for the drawer.

An oven can include connected or wireless refrigerated-cooking feedback tools located inside the refrigerated-cooking compartment, attached to a product or container to be refrigerated or cooked inside a compartment or attached to an open or closed product container which holds the product placed inside the refrigerated-cooking compartment.

Furthermore, a refrigerated oven or microwave can each incorporate numerous operating modes as discussed herein. Each can function to refrigerate and preserve a food item. Each can function to defrost, cook or warm a food item. Each can use UV light treatment to disinfect a food item and incorporate cameras, smart/sensors and readers to track, monitor, view (504) and control the cooking process. Each function can operate individually or together in sequence and additionally each function or mode can be network, locally or remotely controlled and operated by an appliance. Furthermore, product refrigeration, warming, defrost and UV information can be entered into an oven or microwave, as previously noted, via the internet, reading or scanning bar/QR codes or smart/sensors, using voice to activate voice controls to enter said information or using smartphone apps such as reading or scanning smart/sensors or bar/QR codes. A user can individually select each of the following modes, combinations or sequential combinations or said product information can be automatically entered and the user can then edit said information. Any of the technologies disclosed herein can function, operate or be combined in any complementary order or combination.

A smart control or appliance can include voice recognition, interaction and speech using appliance or internet or cloud connected software as discussed herein to allow a user to provide commands or trigger words to control, regulate and activate an appliance or an external appliance for each appliance function such as vacuum, refrigeration, preserve, cook, warm, vent, add or remove products, change meal or predetermined cook times or any of the other appliance modes, functions or processes noted herein including each cooking and preservation function and mode.

A smart control or appliance can also incorporate single or multiple cameras that can be AI connected in a compartment connected and in communication with an appliance control and operating system to identify users, individuals, containers, products, transactions and events using appliance, internet and satellite or cloud software, interfaces and processes as noted herein. For example, a camera can be used to identify a container or a product placed inside a compartment to adjust the required compartment temperature for an individual product or set a specific or mean temperature for more than one product. If the camera does not view a product in a compartment the temperature can be lowered to a predetermined temperature level until a product or container is identified. A camera can identify containers placed into an appliance to activate functions to create a predetermined vacuum level or to vent containers for one or more products with an individual, mean or optimized setting for all items, as with the temperature setting, accessing said data from a an local, remote or networked food data base. A camera can identify food items inside an appliance compartment and set the most effective temperature, humidity, venting and pressure settings for one or more food items that can include the same or different items such as fruit and vegetables, meat, dairy, bakery, fish or a range of respiring or non-respiring food items. A camera can capture images of food during the refrigeration and cooking process to control or regulate the temperature or turn on and off appliance functions if splattering, burning, fire or other identified events occur. A camera can capture a container or food item being placed inside a refrigerated appliance and recommend to a user the most effective storage placement inside the refrigerator by suggesting via voice, light or location the most effective food drawer, shelf, appliance function or location to place said product. A camera can identify a user and recommend food items inside an appliance that fit a described fitness or lifestyle based on health, weight or medical or health needs and requirements. A camera as noted herein can connect to and communicate with a smart/sensor.

Each appliance operating system such as vacuum, temperature, UV, outgassing, venting and humidity can communicate or connect to smart/sensors and said smart/sensors can communicate or connect to operating systems such as vacuum, temperature, UV and humidity systems to activate, deactivate, interrupt, create, regulate and maintain individual or selected operating systems, processes and environments as described herein. For example, smart/sensors such as temperature, vacuum, gas and humidity, can communicate with a control and operating system to individually or in combination activate or deactivate a pump or valve(s) to an open or close position when a predetermined level, range or value of temperature, vacuum, gas, time or humidity is created, maintained or exceeded inside a compartment or inside a container disposed therein or after a preset period of time. A compartment can include a seal (91) on the open and close mechanism or device for the compartment aperture as disclosed herein. All appliance functions and processes can be controlled and monitored via an appliance, smartphone or program or app.

Status indicators can include a sound, noise, light or series of lights located on the front of an appliance which can illuminate in colors to inform of each system's status for any appliance function or mode, such as, red when a vacuum is being created and green when a vacuum is being released from a compartment or when a UV light source treatment is in process or has terminated. Furthermore, a compartment can include an external or internal multi-colored light source that is activated when one of the functions are initiated and additionally can remain activated as long as a product is left or remains inside a compartment or if an appliance aperture has not been opened and closed since the last appliance operation. This light source can have designated light colors for each or unique appliance events; for example, a microwave function can include a red light; a vacuum function a green light; and a UV light treatment a blue light to status the appliance function. Additionally, a combined appliance system can incorporate a default setting, such that an appliance is always in a microwave setting or function, an oven in a refrigeration or cooking mode and other functions such as vacuum sealing or UV light treatment can be selected but then default to the microwave oven function.

As shown herein, an appliance housing (1) can include a compartment (6) disposed therein comprising exterior walls with a slidable drawer (4) that can serve as an internal holding area or retaining compartment (9) for goods with a front wall (10) that can serve as a means to open, close or seal said compartment. Housings can include, but are not limited to, a microwave oven, refrigerator, freezer, cabinet, shell, breadbox, drawer or hinged or unhinged doored or covered compartments. A compartment can include an aperture comprising a drawer (6), door (93), bin, pivot bin, lid or cover, any of which can be hingedly attached to a housing or compartment or surrounding area.

An appliance compartment (6) can be constructed and arranged so that when a retaining compartment, such as a drawer (10), is in a closed position inside a compartment said drawer can create a seal between the engaging surfaces of a compartment and the interior of a drawer. The created seal can also be airtight or can create a vacuum or microwave seal. An induction plate can also be incorporated into a drawer with other appliance functions such as vacuum, etc. Further, in other embodiments, when an appliance vacuum is created inside a compartment a seal can be formed between a front wall of a drawer unit (7) and the walls of a compartment (8). A sealing device (91) can also be provided positioned between or around the engaging surfaces of a compartment and the interior (9) of a drawer or a door (93). Additionally, a sensor (12) can be positioned on a drawer or door of a refrigerator or compartment and connect to operating systems and to a control system to indicate the open, close and seal status to activate and deactivate individual or selected operating systems. Furthermore, a mechanical or electrical latch mechanism can be provided to secure a drawer or aperture and a compartment together when the interior portion of a drawer is substantially contained inside a compartment to engage a drawer and a compartment together to seal said compartment interior. A latch mechanism can connect to a control system and to individual or selected operating systems to activate or deactivate individual or selected operating systems. A drawer can also include a handle (5).

Other aspects of the invention provide an appliance programmable control system, which can comprise a display panel (41), operating system controls (42) and a microprocessor and memory device (92), for a housing or a compartment to connect and communicate with operating system components to activate, deactivate, control, monitor, release and regulate any of the operating and elements and systems disclosed herein such as a pump(s) (43), latch mechanism, sensors (50), valves (48), system status indicators connected to sensors, and controls, and microwave oven, oven, induction plate, external appliances, UV light source, vacuum, temperature, outgassing and humidity systems. Accordingly, each operating system and compartment can interconnect and inter-communicate with each other, or via a control system, to function together or individually to carry out the processes described herein. Accordingly, as an example, a control system can activate or deactivate a pump to vent, cool or create vacuum and pressure environments inside a compartment, as well as, activate or deactivate a gas or release valve to open or close positions to seal or unseal a compartment. Additionally, a control system can connect to and communicate with smart/sensors (50) described herein and said smart/sensors can connect to and communicate with a control system (20) and each operating system such as a pump(s), valves, latch mechanism, smart/sensors, system status indicators, system controls and vacuum, temperature and humidity systems to activate, deactivate, create, monitor, regulate, maintain and release operating systems, environments, such as, vacuum, pressure, temperature, humidity, gas, air, components and processes for and in a housing, compartment and containers disposed therein and combinations thereof. A user interface with an led display and apparatus power source (46, 47) with touch, keyed or digital display panel functionality can operatively connect to and communicate with a control system to allow the input of instructions, formulas, time sequences, algorithms and values to operate external appliances and each or selected appliance system components, systems and processes described herein. A user interface, situated on a compartment or a housing exterior or interior front panel, access or surrounding areas can also comprise touch panel controls, switches, buttons and knobs to connect to and communicate with operating systems and housing components. The operating systems can be programmed to be activated and deactivated automatically via a control system or manually via a control panel or via each operating system.

In another embodiment a vacuum sensor can communicate with a compartment interior and operatively connect to a housing, compartment or control system to open and close a gas or release valve and to activate and deactivate a pump(s). Alternately, a vacuum sensor can connect directly to a pump(s), a housing, compartment or valve. Furthermore, a housing or compartment sensor, as described herein, such as vacuum, temperature, pressure, tactile, drawer and refrigerator door open, close and seal status sensors can operatively connect in combination to a pump, valve(s), sensors and housing or compartment operating systems to open and close a valve(s) or to activate or deactivate a pump(s).

A control system or pump and valve can also be activated to vent or cool a compartment interior by activating a pump to draw or pump air through a compartment interior and contemporaneously activate a valve or a release valve to open positions. These functions can be activated simultaneously with other appliance modes such as microwave, induction plate and compartment heat element functions. A compartment or housing control system or a vacuum sensor can activate a pump and a valve to create a predetermined vacuum level in a compartment or container disposed therein. A pressure release or valve can release a predetermined amount of vacuum from inside a compartment at a predetermined time period, pressure level or when a level is reached or exceeded and a vacuum can also be released via the aperture of a compartment.

A compartment can comprise multiple storage functions or modes (45) such as, but not limited to, a conventional compartment with operating systems placed in either off or idle positions, a vacuum compartment, a pressurized compartment, a venting compartment, a cooling compartment, a humidity compartment, an outgassing compartment, a processing compartment to create environments inside containers disposed therein, such as temperature, humidity, vacuum, pressure, venting, cooling and outgassing, a processing compartment to emit UV light to treat items placed inside said compartment, a monitoring compartment to view, monitor and interact with items inside said compartment via a camera located inside said compartment, a microwave oven, induction plate, electrical heating element and an oven function to defrost or cook items placed inside said compartment, among others.

Some of the appliance, operating and control system functionalities and modes can include, but are not limited to:

1.) A function to create a range of temperature inside a housing to cool or warm a compartment or container disposed therein.

2.) A function to activate a pump and close a valve or to close a valve and activate a pump or to contemporaneously activate a pump and close a valve to create a vacuum environment inside a compartment when said compartment aperture is closed and sealed.

3.) A function to activate a pump and close a valve or to close a valve and activate a pump or to contemporaneously activate a pump and close a valve to create a pressurized environment inside a compartment when said compartment aperture is closed and sealed.

4.) A function to activate a pump (in vacuum or pressure mode) while a compartment valve is contemporaneously actuated to an open position to vent, cool or degas said compartment and contents therein. Furthermore, a temperature smart/sensor or control system can activate a valve to a close position after a predetermined value, time period or level of vacuum, temperature or humidity has been established, reached or exceeded inside a compartment. This setting is applicable to any mode or function described herein.

5.) A function to activate a pump (vacuum or pressure mode) and to simultaneously close a valve to create a pressure or vacuum environment inside a compartment and to maintain said pressure or vacuum level inside said compartment until it is deactivated manually or automatically via a control system.

6.) A function to activate a pump and to close a valve to create a pre-determined vacuum in a compartment and when said maximum pressure level reaches or exceeds a pre-determined level, time period or value to then deactivate said pump and open a valve to release said vacuum and open said compartment by admitting air into said compartment.

7.) A function to activate a pump and to close a valve to create a preset vacuum level inside a compartment and when said maximum vacuum level reaches or exceeds a pre-determined time period, level or value to open a valve to release said vacuum by admitting air into a compartment and to deactivate said pump.

8.) A function to activate a pump and to close a valve to create a vacuum inside a compartment and when said maximum vacuum level reaches or exceeds a pre-determined level, time period or value to concurrently open a valve and deactivate a pump to admit air into said compartment to release said vacuum.

9.) A function to activate a pump and to close valves to create a preset vacuum in a compartment and thereafter for a pump to intermittently remove air or gas from the interior of said compartment or a container disposed therein. Further, said pump can intermittently remove air and gas following the reduction of the pressure in a compartment to a selected pressure. As previously noted, a smart/sensor as described herein, can communicate or connect to an appliance, a compartment interior, a container, a product, a control system or operating system such that when a predetermined gas level fails, reaches or exceeds a pre-determined level or value smart/sensor can communicate with an appliance control system, activate a pump to remove additional air or gas from a compartment or send a notice to a client device, user, network, local or remote computing device or third party service/product provider. Further, a smart/sensor can communicate with a control system, activate a pump or activate a valve to an open position to remove additional air or gas from a compartment. A smart/sensor can also activate a pump and activate a valve to an open position to vent a compartment and remove air or gas, including a container disposed therein. As disclosed herein, smart/sensors can be located in product storage areas, appliances, containers or attached or affixed to appliances and function as previously noted.

10.) A function to activate a pump and to close a valve to create a vacuum in a compartment, maintain said vacuum level for a pre-determined period of time and, thereafter, open a valve to admit air into said compartment to release a vacuum.

11.) A function to activate and place a valve in a closed or open position to seal or unseal a compartment to create, regulate and release environments from inside a compartment.

12.) A function to concurrently open a valve and to activate a pump (vacuum or pressure mode) to draw or push air into or out of a compartment to release or create environments therein.

13.) A function to deactivate a pump when a valve is in a close position to admit air into a compartment through an open and close aperture mechanism, such as a drawer or door opening, of a compartment to release a vacuum.

14.) A function to deactivate a pump and admit air into a compartment through an open and close aperture mechanism, such as a drawer or door opening, of a compartment to release a vacuum.

15.) A function to deactivate all or selected operating systems or place all or selected operating systems in an idle state.

16.) A function to close or open a valve when a compartment aperture mechanism, such as a drawer or door opening, is in a close or open position via an aperture sensor placed on a compartment or refrigerator aperture and in communication with a control system.

17.) A function to allow a valve to have an independent pressure setting or to be connected to a control system in a compartment to automatically open to release a vacuum created therein when a vacuum level is reached or exceeded inside said compartment and to automatically deactivate a vacuum pump creating said vacuum. As used herein, when a vacuum is released from a compartment said compartment is returned to atmospheric pressure.

18.) A function to operate, control and regulate a single or multiple UV lights positioned inside a compartment to treat the environment, containers or food items placed therein. The UV light can function in an appliance storage function or container processing mode and can further be sequenced to function for a period of time, a value or time interval prior to, in one instance, creating and maintaining a vacuum environment. Furthermore, the UV lighting treatment can function intermittently for programmed or sequenced time intervals or periods or upon the certain environmental parameters being reached and between and in sequence with other appliance functions.

19.) A function or mode to operate and control a microwave oven or oven to defrost, prepare and cook items placed inside a compartment.

20.) A function or mode to operate and control a camera for facial recognition, product recognition and event recognition using still photos and video and connected and in communication with voice recognition and interaction systems as well as AI, event, product and facial recognition software and internet and cloud connectivity to monitor and interact with users, events and products.

21.) A function for all modes to allow a control system or a container with a wireless communication device to automatically activate a mode, series or sequence of modes or functions for determined or sensor prompted actions as described herein.

22.) A function that automatically turns off the temperature or reduces said temperature to a predetermined level when no container or food item is detected inside an appliance or compartment when the temperature function is activated and then return the temperature level to the previous setting when a food item or container is placed, identified or sensed inside said compartment. This function can be selectively activated or automatically via a sensor in a compartment or a sensor and wireless device attached to or inside a container in communication with a control system or via a camera system described herein connected to AI, internet and cloud services.

23.) A function or mode to activate, deactivate, regulate and operate a vacuum hose attachment to create vacuum environments inside one-way valve containers and bags. Said function can be selectively activated via a switch connected and in communication to the vacuum hose pump or via an appliance control system connected and in communication with the vacuum pump.

24.) A function or mode to activate, deactivate, regulate and operate a handheld

UV light to disinfect and treat an appliance compartment or food and items placed inside the appliance compartment. The UV light can connect and communicate with an appliance control system or can connect, communicate and be actuated via a switch. Furthermore, a UV light can be wireless, rechargeable and be attached to, located or situated inside an appliance, appliance housing or compartment such as a recess or other constructed area to hold said device.

25.) An appliance function or mode to control, regulate and operate a humidity system to humidify the interior of an appliance compartment and containers placed therein.

26.) An appliance function or mode to control, regulate and operate an induction plate connected to or in communication with an appliance.

27.) An appliance function or mode to control, regulate and operate the temperature of an electrical heating element inside an appliance or compartment to function individually or in combination for example with an induction plate.

28.) An appliance function or mode to calculate, control, regulate and operate an appliance software and hardware program to control the simultaneous cooking sequence and timing of single or multiple external appliances connected to and in communication with an appliance as well as the host appliance with wireless connectivity to internet and cloud functions to program, time and sequence multiple appliance cooking times and functions.

29.) An appliance function or mode to electrically (mechanically) open and close an appliance compartment venting system to open to allow compartment venting and cooling and to close to create a sealed or vacuum sealed environment.

30.) An appliance or smart control function or mode incorporating an AI connected camera with product, image, facial, transactional, event and activity recognition software to monitor an appliance housing, compartment and areas in the vicinity of an appliance, compartment or smart control.

Further, methods are provided for the use and interaction of a smart control, appliance and smart power-strip network and individually, as well as, for the use of containers, described herein, for use with the network and individually for a smart control and appliance and for the operation and function of each container.

Control and operating systems, elements, functions or modes disclosed herein can be connected to or in communication with said appliance control and operating system which in turn can be connected to internet and cloud services. The appliance control system can be pre-programmed or programmable for each individual mode or operating function or combinations thereof. For example, each mode can be manually, automatically or in combination, selected via an appliance control system or each operating system selectively or individually, in combination or sequence. Single or multiple appliance modes or functions can sequence together, simultaneously, in series, selectively or automatically, and upon predetermined, programmable, sensed, timed, heard, voice, triggered, camera viewed, AI motivated or acted upon or environmental parameters not achieved, achieved or exceeded. For example, a venting mode can be selected to vent a compartment at specified time or gas intervals and during said operation a user can place a one-way valve container into said compartment and select a vacuum sealing mode or the container can wirelessly communicate this mode to the control system to activate. The vacuum sealing mode can be activated and once a predetermined vacuum level is created and released this mode terminates and the venting mode automatically resumes. Further, each of the appliance modes can operate and function via a predetermined level, range or value of temperature, vacuum, gas, time, interval, occurrence or humidity level is created, maintained or exceeded inside a compartment or inside a container disposed therein or after a preset period of time. A compartment can also include sensors, such as weight, laser, proximity or other, connected to and in communication with said containers or control system to sense or identify if a food item or container is present inside a compartment when said temperature function has been activated and requires the temperature function. If a food item or container is sensed then the temperature function can be activated and maintained. If a food item or container is not sensed or identified then the temperature setting can be turned off or lowered to a preset temperature setting to save on the appliance operating costs. Each of the appliance functions and modes described herein include methods for operations and functions described herein such as selecting an appliance function or mode, combining appliance functions and modes, using a container with appliance functions and modes, combining said functions and modes and sequencing appliance functions or modes with other functions and modes and with functions and modes of a smart control, smart power-strip or appliance which can be further defined by appliance environmental, operational and control systems and internet, cloud, AI, product, event and facial recognition software and voice command and recognition connected parameters.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A system for identifying a food product in a container in a refrigerator and determining a stimulus or a level of freshness, spoilage or contamination of the food product, comprising:
a radio frequency tag inside the container or connected to the container;
a computing device configured to communicate with a user interface device, the computing device comprising a processor and a non-volatile, non-transitory memory;
a radio frequency reader in communication with the computing device, the radio frequency reader configured to send a radio wave to the container and the radio frequency tag, and receive a signal from the radio frequency tag;
the radio frequency tag is configured to send identity information about the food product or the container to the computing device;
the radio frequency tag is configured to receive the radio wave from the radio frequency reader to power the radio frequency tag;
the radio frequency tag comprising a sensor portion that contacts or interacts with the stimulus when present in the container, wherein the stimulus comprising a solid compound, a liquid compound, or a gaseous compound from the food product or a contaminate in the container;
the radio frequency tag sending a signal to the radio frequency reader, the signal containing information about the stimulus measured by the sensor;
an application stored in the non-volatile, non-transitory memory and executable by the processor to:
identify the food product or the container based on the identity information received from the radio frequency reader and send the identity of the food product or the container to the user interface device;
interpret the signal to detect the stimulus or to determine a level or quantity of the stimulus;
determine at least one of, the stimulus or that the food product is spoiled, the food product is contaminated or a level of freshness of the food product based on whether the level or quantity of the stimulus reaches or exceeds a profile; and
send to the user interface device a notice of the detection of the stimulus or that the food product is spoiled, the food product is contaminated, or a level of freshness of the food product.

2. The system of claim 1, wherein the radio frequency tag comprises a radio frequency identification tag, an NFC tag, or a radio frequency tag configured to use a Bluetooth protocol and the radio frequency reader and the computing device are configured to read and interpret the signal containing information about the stimulus measured by the sensor from the radio frequency identification tag, the NFC tag or the tag using a Bluetooth protocol.

3. The system of claim 1, wherein the stimulus comprises one of an analyte, a mold, an ethylene, an alkene, an alkyne, an acid, a ketone, an ester, an aldehyde, an alcohol, an ether, a thiol, an ammonia, mono-nitrogen oxide or an amine.

4. The system of claim 1, wherein the user interface device comprises at least one of a smart speaker, a smartphone, a handheld device, a client device, a tablet, a beacon or a display to display information.

5. The system of claim 1, wherein the application further comprising transmitting an order request or placing the food product on a digital list.

6. The system of claim 5, wherein the order request or the digital list comprises a purchase, an order, a replacement, or inventory management for the food product for a manufacturer, a wholesaler, a retailer, order chain, or a consumer.

7. The system of claim 1, wherein the radio frequency tag is attached to an exterior of the container.

8. A system for identifying a food product in a container and determining a stimulus or a level of freshness, spoilage or contamination of the food product with an appliance, comprising:
a radio frequency tag inside the container or connected to the container;
an appliance computing device comprising a processor and a non-volatile, non-transitory memory;
a radio frequency reader in communication with the computing device, the radio frequency reader configured to send a radio wave to the container and the radio frequency tag, and receive a signal from the radio frequency tag;
the radio frequency tag is configured to send identity information about the food product or the container to the computing device;
the radio frequency tag is configured to receive the radio wave from the radio frequency reader to power the radio frequency tag;
the radio frequency tag comprising a sensor portion that contacts or interacts with the stimulus when present in the container, wherein the stimulus comprising a solid compound, a liquid compound, or a gaseous compound from the food product or a contaminate in the container;
the radio frequency tag sending a signal to the radio frequency reader, the signal containing information about the stimulus measured by the sensor;
an application stored in the non-volatile, non-transitory memory and executable by the processor to:
identify the food product or container based on the identity information received from the radio frequency reader and send the identity of the food product or the container to the appliance;
interpret the signal to detect the stimulus or to determine a level or quantity of the stimulus;
determine at least one of, the stimulus or that the food product is spoiled, the food product is contaminated or a level of freshness of the food product based on whether the level or quantity of the stimulus reaches or exceeds a profile; and
send to the appliance a notice of the detection of the stimulus or that the food product is spoiled, the food product is contaminated, or a level of freshness of the food product.

9. The system of claim 8, wherein the radio frequency tag comprises a radio frequency identification tag, an NFC tag, or a radio frequency tag configured to use a Bluetooth protocol and the radio frequency reader and the computing device are configured to read and interpret the signal containing information about the stimulus measured by the sensor from the radio frequency identification tag, the NFC tag or the tag using a Bluetooth protocol.

10. The system of claim 8, wherein the stimulus comprises one of an analyte, a mold, an ethylene, an alkene, an alkyne, an acid, a ketone, an ester, an aldehyde, an alcohol, an ether, a thiol, an ammonia, mono-nitrogen oxide or an amine.

11. The system of claim 8, wherein the appliance comprises one of a smart speaker, a tablet, a smartphone, a beacon, a handheld device, a client device, a server, a display to display information, or a refrigerator.

12. The system of claim 8, wherein the application further comprising transmitting an order request or placing the food product on a digital list.

13. The system of claim 12, wherein the order request or the digital list comprises a purchase, an order, a replacement, or inventory management for the food product for a manufacturer, wholesaler, retailer, order chain, or consumer.

14. The system of claim 1, wherein the radio frequency tag is attached to an exterior of the container.

15. A method for identifying a food product in a container in a refrigerator and determining a stimulus or a level of freshness, spoilage or contamination of the food product, comprising:
providing a radio frequency tag inside the container or connected to the container, a computing device configured to communicate with a user interface device, the computing device comprising a processor and a non-volatile, non-transitory memory, a radio frequency reader in communication with the computing device, the radio frequency reader configured to send a radio wave to the container and the radio frequency tag, and receive a signal from the radio frequency tag, the radio frequency tag is configured to send identity information about the food product or the container to the computing device; the radio frequency tag is configured to receive the radio wave from the radio frequency reader to power the radio frequency tag, the radio frequency tag comprising a sensor portion that contacts or interacts with the stimulus when present in the container, wherein the stimulus comprising a solid compound, a liquid compound, or a gaseous compound from the food product or a contaminate in the container; the radio frequency tag sending a signal to the radio frequency reader, the signal containing information about the stimulus measured by the sensor, and an application stored in the non-volatile, non-transitory memory and executable by the processor to:
identifying the food product or container based on the identity information received from the radio frequency reader and sending the identity of the food product or the container to the user interface device;
interpreting the signal to detect the stimulus or to determine a level or quantity of the stimulus;
determining at least one of, the stimulus or that the food product is spoiled, the food product is contaminated or a level of freshness of the food product based on whether the level or quantity of the stimulus reaches or exceeds a profile; and
sending to the user interface device a notice of the detection of the stimulus or that the food product is spoiled, the food product is contaminated or a level of freshness of the food product.

16. The system of claim 15, wherein the radio frequency tag comprises a radio frequency identification tag, an NFC tag, or a radio frequency tag configured to use a Bluetooth protocol and the radio frequency reader and the computing device are configured to read and interpret the signal containing information about the stimulus measured by the sensor from the radio frequency identification tag, the NFC tag or the tag using a Bluetooth protocol.

17. The system of claim 15, wherein the stimulus comprises one of an analyte, a mold, an ethylene, an alkene, an alkyne, an acid, a ketone, an ester, an aldehyde, an alcohol, an ether, a thiol, an ammonia, mono-nitrogen oxide or an amine.

18. The system of claim 15, wherein the user interface device comprises at least one of a smart speaker, a smartphone, a handheld device, a client device, a tablet, a beacon or a display to display information.

19. The system of claim 15, wherein the application further comprising transmitting the order request or placing the food product on a digital list.

20. The system of claim 19, wherein the order request or the digital list comprises a purchase, an order, a replacement, or inventory management for the food product for a manufacturer, wholesaler, retailer, order chain, or consumer.
